(19) United States
(12) Patent Application Publication
(10) Pub. No.: US 2002/0068287 A1
(43) Pub. Date: Jun. 6, 2002

(54) Methods of identifying integrin ligands using differential gene expression

(76) Inventors: Antonin de Fougerolles, Brookline, MA (US); John Carulli, Southborough, MA (US); Victor Kotelianski, Boston, MA (US); Cynthia D. Green, Madison, CT (US); Andro Hsu, Berkley, CA (US)

Correspondence Address: MINTZ, LEVIN, COHN, FERRIS,, GLOVSKY and POPEO, P.C., One Financial Center, Boston, MA, 02111, US

(21) Appl. No.: 09/825,522

(22) Filed: Apr. 2, 2001

Related U.S. Application Data
(60) Provisional application No. 60/193,629, filed on Mar. 31, 2000.

(51) Int. Cl. C12Q 1/68; C07H 21/02; C07H 21/04; C12P 19/34

(52) U.S. Cl. 435/6; 435/91.2; 536/23.1

(57) ABSTRACT

Disclosed are methods of identifying integrin modulating agents using differential gene expression. Also disclosed are novel nucleic acid sequences whose expression is differentially regulated by treatment with extracellular matrix components or growth factors.

### RELATED U.S. APPLICATION

This application claims priority to U.S. Ser. No. 60/193,629, filed Mar. 31, 2000 which is incorporated herein by reference in its entirety.

### FIELD OF THE INVENTION

The invention relates generally to nucleic acids and polypeptides and in particular to the identification of integrin modulating agents using differential gene expression.

### BACKGROUND OF THE INVENTION

Integrins are cell surface receptors that are evolutionarily conserved molecules found in a wide variety of cell types. They are composed of two subunits, an alpha and a beta subunit, and there are many varieties of each subunit. These subunits are are mixed and matched in various cell types to form specialized receptors that have unique binding specificity and signaling capabilities. Specialized signaling via integrins effects the activities of cytoplasmic kinases, growth factor receptors, ion channels and organization of the intracellular cytoskeletal components. These activities can direct cell proliferation, differentiation, or apoptosis, cell fates which are important in many aspects of normal growth as well as disease. Giancotti and Ruoslahti, 1999 Science 285: 1028-1032. Integrins are also important for optimal function of other membrane receptors such as PDGF, EGF and VEGF, under certain attachment conditions.

### SUMMARY OF THE INVENTION

The invention is based in part on the discovery that certain nucleic acids are differentially expressed in monocytes when exposed to various extracellular matrix components in the presence or absence of growth factors.

In further aspect, the invention provides a method of screening a test agent for integrin modulating activity. For example, in one aspect, the invention provides a method of identifying a integrin modulating agent by providing a test cell population comprising a cell capable of expressing one or more nucleic acids sequences responsive to integrin modulators, contacting the test cell population with the test agent and comparing the expression of the nucleic acids sequences in the test cell population to the expression of the nucleic acids sequences in a reference cell population not treated with an integrin modulators. An alteration in expression of the nucleic acids sequences in the test cell population compared to the expression of the gene in the reference cell population indicates that the test agent is an integrin modulator.

Also provided are novel nucleic acids, as well as their encoded polypeptides, whose expression is responsive to the effects of integrin modulating agents.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention is based in part on the discovery of changes in expression patterns of multiple nucleic acid sequences in the human monocytic cell line, THP-1 following exposure to various integrin modulating agents. The intergin modulating agents included extracellular matrix components, e.g., fibronectin, type I collagen, laminin and VCAM.

The differentially expressed nucleic acids were identified by culturing the THP-1 cells either adherently or in suspension in the presence of the integrin modulating agent. Control cultures received poly-lysine. Following treatment cells were lysed and total RNA was recovered from the lysed cells. cDNA was prepared and the resulting samples were processed through using GENECALLING™ differential expression analysis as described in U.S. Pat. No. 5,871,697 and in Shimkets et al., Nature Biotechnology 17: 798-803 (1999). The contents of these patents and publications are incorporated herein by reference in their entirety.

Thousands of gene fragments were initially found to be differentially expressed in THP-1 cells in response to integrin modulating agents. Genes fragments whose expression levels were modulated greater than ±2.0-fold were selected for further analysis.

A summary of the sequences analyzed are presented in Tables 1-3. The 260 single nucleic acid sequences identified herein, are referred to herein as KEANOX 1-260.

Twenty-five sequences (KEANOX: 1-25) represent novel rat genes for which the sequence identity to sequences found in public databases suggesting a putative homology.

The 235 other sequenced identified have been previously described. For some of the novel sequences (ie., KEANOX: 1-25), a cloned sequence is provided along with one or more additional sequence fragments (e.g., ESTs or contigs) which contain sequences substantially identical to, the cloned sequence. Also provided is a consensus sequences which includes a composite sequence assembled from the cloned and additional fragments. For a given KEANOX sequence, its expression can be measured using any of the associated nucleic acid sequences may be used in the methods described herein. For previously described sequences database accession numbers are provided. This information allows for one of ordinary skill in the art to deduce information necessary for detecting and measuring expression of the KEANOX nucleic acid sequences.

The integrin modulating agent responsive nucleic acids discussed herein include the following:

TABLE 1
Novel Nucleic Acids

| | SEQ ID | KEANOX | Integrin Modulator Effect of Transcript Level | | | | | | | |
|---|---|---|---|---|---|---|---|---|---|---|
| Sequence Description | NO | Assign. | 4798 | 4799 | 4800 | 4801 | 4802 | 4865 | 5552 | 5553 |
| ai150417: qf35f04.xl Soares_testis_NHT | 1 | 1 | — | — | — | — | — | — | 8 | — |
| Homo sapiens cDNA clone IMAGE: 1752031 | | | | | | | | | | |
| 3′, mRNA sequence-Homo sapiens, 366 bp | | | | | | | | | | |
| (RNA). [N] | | | | | | | | | | |
| cghsb0n0127.1_3: novel fragment [C] [N] | 2 | 2 | −2 | −8.3 | — | −5.1 | — | — | — | — |
| cghsblr0148.6_51: same as AC000353 | 3 | 3 | — | — | — | 4.9 | — | — | — | — |
| chromosome 11q13 sequence (95% over 149 | | | | | | | | | | |
| bp) [N] | | | | | | | | | | |
| cghsi0a056.4_3: novel fragment [N] | 4 | 4 | — | — | — | — | −5.1 | 4 | — | — |
| cghsi0r0299.6_10: sim. to Human factor XIII | 5 | 5 | — | — | 6.2 | — | 3.3 | — | — | — |
| b subunit gene m64554 (87% identical over | | | | | | | | | | |
| 161 bp) [N] | | | | | | | | | | |
| cghs10h0354.8_1: sim. to X69878 H. sapiens | 6 | 6 | — | — | — | — | — | — | 3.9 | — |
| Flt4 mRNA for transmembrane tyrosine | | | | | | | | | | |
| kinase (85% identical over 334 bp) [N] | | | | | | | | | | |
| cghs10n0322.1_1: some sim. to Human simple | 7 | 7 | — | −2.4 | — | −2.4 | −11.3 | 8.1 | — | — |
| repeat polymorphism M87670 (57% identical | | | | | | | | | | |
| over 185 bp) [N] | | | | | | | | | | |
| cghsmli0276.4_3: novel sequence [N] | 8 | 8 | — | — | — | — | 9.2 | — | — | — |
| cghsp0t0125.4_12: novel fragment (sim. to rpt. | 9 | 9 | — | — | — | 5.1 | — | — | — | — |
| regions) [N] | | | | | | | | | | |
| cghsr0c0202.5_1: sim. to many repeat regions | 10 | 10 | — | — | — | — | −5.3 | — | — | — |
| cghsr0s0131.1_4801-186: overlaps w/ | 11 | 11 | 1.6 | 3.1 | — | 3.1 | — | −2.3 | — | — |
| patented sequence x27375 [N] | | | | | | | | | | |
| cghsr0w0118_4801-98: novel gene fragment | 12 | 12 | −1.8 | −2.3 | — | −4.2 | — | 2.8 | — | — |
| cghss011157.5_4865-113: novel fragment [N] | 13 | 13 | — | — | — | 1 7 | — | −2.8 | — | — |
| cghss0v0354.7_3: identical to chromosome | 14 | 14 | — | — | — | — | — | — | −4.3 | — |
| 21q11.1 DNA segment 12/28 (AP000041) [N] | | | | | | | | | | |
| cghsy0h0150.9_4801-109: novel fragment [N] | 15 | 15 | — | 5 | — | 4.6 | — | −2.4 | — | — |
| cghsy0i0417.5_1: novel EST; mz5003 FLC; | 16 | 16 | — | — | — | — | −6.6 | 4.3 | — | — |
| overlap with may ESTs, formerly specified as | | | | | | | | | | |
| AA702742 [C] [N] | | | | | | | | | | |
| cghsy0k0280.8_4801-59: novel gene fragment | 17 | 17 | — | 1.9 | — | 2.4 | — | — | — | — |
| cgn011hs55.3_2: identical to a1022240 Human | 18 | 18 | — | — | — | — | −3.4 | 2.9 | — | — |
| DNA sequence in progress from clone 328E19 | | | | | | | | | | |
| (104815 bp) [N] | | | | | | | | | | |
| | | | 5554 | 6551 | 6552 | 6554 | 6556 | 6890 | 6891 | 6892 |
| ai073404: ov46b03.x1 Soares_testis_NHT | 19 | 19 | — | — | — | −5.4 | −2.7 | — | — | −3.3 |
| Homo sapiens cDNA clone IMAGE: 1640333 | | | | | | | | | | |
| 3′, mRNA sequence-Homo sapiensp, 585 bp | | | | | | | | | | |
| (RNA). [N] | | | | | | | | | | |
| ai088930: qa21g03.x1 NCI_CGAP_Bm23 | 20 | 20 | — | 7.4 | 4.1 | — | 5.7 | — | — | 2 1 |
| Homo sapiens cDNA clone IMAGE: 1687444 | | | | | | | | | | |
| 3′, mRNA sequence-Homo sapiensp, 553 bp | | | | | | | | | | |
| (RNA). [N] | | | | | | | | | | |
| ai275149: q170h12.x1 Soares_NhHMPu_S1 | 21 | 21 | — | — | — | 6.8 | 5.3 | — | — | 3.1 |
| Homo sapiens cDNA clone IMAGE: 1877735 | | | | | | | | | | |
| 3′, mRNA sequence-Homo sapiensp, 470 bp | | | | | | | | | | |
| (RNA). | | | | | | | | | | |
| ai571533: tr85c12.x1 NCI_CGAP_Pan1 | 22 | 22 | — | — | — | — | — | −17 | −21 | — |
| Homo sapiens cDNA clone IMAGE: 2225110 | | | | | | | | | | |
| 3′, mRNA sequence-Homo sapiensp, 492 bp | | | | | | | | | | |
| (RNA). [N] | | | | | | | | | | |
| ai591110: tw91d04.x1 NCI_CGAP_HN6 | 23 | 23 | — | — | — | — | 4.3 | — | — | — |
| Homo sapiens cDNA clone IMAGE: 2267047 | | | | | | | | | | |
| 3′, mRNA sequence-Homo sapiensp, 490 bp | | | | | | | | | | |
| (RNA). [N] | | | | | | | | | | |
| cghsmli0276.4_3: novel sequence [N] | 24 | 24 | — | — | — | −7.6 | −7.5 | — | — | — |
| novel fragment: novel fragment overlaps with | 25 | 25 | 2.5 | — | — | 3.5 | 4.9 | — | — | 5.6 |
| ESTs [N] | | | | | | | | | | |

Key to treatment groups:
4798 = collagen type I
4799 VCAM
4800 = laminin
4801 = fibronectin
4802 = fibronectin
4865 = poly lysine
5552 = VCAM
5553 = fibronectin
6892 = VCAM
5554 = polylysine
6551 = fibronectin
6552 = VCAM
6554 = polylysine
6556 = fibronectin
6890 = fibronectin
6891 = VCAM

TABLE 2
Known Genes

| | | Integrin Modulator Effect of Transcript Level | | | | | | | | |
|---|---|---|---|---|---|---|---|---|---|---|
| | GenBank | KEANOX | | | | | | | | |
| Description of Sequences | Acc. No. | Assign | 4798 | 4799 | 4800 | 4801 | 4802 | 4865 | 5552 | 5553 |
| 01.01.03 mRNA Splicing | | | | | | | | | | |
| SRM: 160: Homo sapiens Ser/Arg-related nuclear | gbh_at048977 | 26 | −2.6 | — | 2.2 | — | −4.7 | 3.5 | — | — |
| matrix protein (SRM160) mRNA, | | | | | | | | | | |
| 01.01.04 Regulators | | | | | | | | | | |
| TSC22: Human TGF-beta stimulated clone 22 | gbh_u35048 | 27 | — | — | — | — | — | — | 2.4 | — |
| homolog | | | | | | | | | | |
| 01.02.01 Ribosomal Proteins | gbh_u14973 | | | | | | | | | |
| RPL18: Homo sapiens 60S ribosomal protein L18 | gbh_111566 | 28 | — | — | −2.3 | −2.1 | — | — | — | — |
| RPS10: Human 40S ribosomal protein S10 | | 29 | — | — | −5.8 | — | — | — | — | — |
| PRS2 OR RPS4: Human 40S ribosomal protein S2 | gbh_x17206 | 30 | — | — | — | 1.4 | −2.8 | −43.5 | — | — |
| (aka LLRep3 protein, S4) | | | | | | | | | | |
| 01.02.02 Amino Acyl tRNA Synthetases | | | | | | | | | | |
| DARS: Human aspartyl-tRNA synthetase | gbh_j05032 | 31 | — | — | −2.2 | — | −3 | — | — | — |
| 18s rRNA: Human 185 rRNA | m10098 | 32 | — | — | — | — | — | — | — | −2 |
| 01.03.01 Molecular Chaperone | | | | | | | | | | |
| HSPCA or HSPC1 or HSP90A: Homo sapiens heat | gbh_m27024 | 33 | — | 3.6 | 3.5 | 2.9 | −2.1 | — | — | — |
| shock protein HSP 90-alpha | | | | | | | | | | |
| p60 EBI3-assoc. protein: Human EBI3-associated | gbh_u41806 | 34 | — | −3 9 | — | −2.9 | −7.7 | 2.2 | 2.7 | — |
| protein p60 (aka phosphotyrosine independent ligand | | | | | | | | | | |
| p62 for the LCK SH2 domain) | | | | | | | | | | |
| p60 EBI3-assoc. protein: Human EBI3-associated | gbh_u46751 | 35 | — | −3.9 | — | −2.9 | −7.7 | 2.5 | — | — |
| protein p60 (aka phosphotyrosine independent ligand | | | | | | | | | | |
| p62 for the LCK SH2 domain) | | | | | | | | | | |
| p60 EBI3-assoc protein: Human phosphotyrosine | gbh_u46752 | 36 | — | −3.9 | — | −2.9 | −7.7 | 9.9 | — | — |
| independent ligand p62B B-cell isoform for the LCK | | | | | | | | | | |
| SH2 domain (aka EBI3-associated protein p60) | | | | | | | | | | |
| 01.05.01 Proteolysis | | | | | | | | | | |
| CAPN4 or CAPNS: Human mRNA for calcium- | gbh_x04106 | 37 | — | — | — | 2 | — | 2.7 | — | 2.2 |
| dependent protease, small subunit (aka calpain | | | | | | | | | | |
| regulatory subunit) | | | | | | | | | | |
| 01.05.02 Ubiquination | | | | | | | | | | |
| G1P2 OR UCRP OR ISG15: Human ubiquitin cross- | uehsf_{—} | 38 | −2.7 | −2.1 | — | −2.3 | −7.8 | 3.2 | — | −1.8 |
| reactive protein (aka interferon-induced 15/17 kDa | 41281_1 | | | | | | | | | |
| protein) | | | | | | | | | | |
| 02 Signal Transduction | | | | | | | | | | |
| TANK: Human TRAF family member-associated NF- | gbh_u63830 | 39 | — | — | — | — | −3.7 | 4.5 | — | — |
| kB activator | | | | | | | | | | |
| RHOA OR RHO12: Human transforming protein rhoA | gbh_x05026 | 40 | — | −6 | — | −2.6 | −3.1 | — | — | — |
| (aka H12) | | | | | | | | | | |
| IL1RN OR IL1RA: H. sapiens interleukin-1 receptor | gbh_x64532 | 41 | −2 | — | — | — | — | — | 8 | −2.4 |
| antagonist protein | | | | | | | | | | |
| 02.01.02 Growth Factors | | | | | | | | | | |
| HEGFL Human heparin-binding EGF-like growth | gbh_m60278 | 42 | — | — | — | −2 | −2.7 | 5.3 | 5.4 | — |
| factor mRNA, complete eds. | | | | | | | | | | |
| CTGF: Human connective tissue growth factor | gbh_m92934 | 43 | — | 4 | — | 2.7 | — | — | — | −1.8 |
| CTGF: Human connective tissue growth factor mRNA, | gbh_u14750 | 44 | — | 5.2 | — | 4.8 | — | −2.6 | 6.8 | −1.8 |
| partial cds. | | | | | | | | | | |
| INHBA: H. sapiens activin beta-A subunit (exon 2). | gbh_x57579 | 45 | — | — | — | — | — | — | 5.6 | — |
| 02.01.03 Cytokines | | | | | | | | | | |
| PBEF: Pre-B Cell Enhancing factor U02020 (129050 | gbh_129050 | 46 | — | −2.3 | — | −2.1 | — | 4.2 | 3.4 | — |
| annotated as GOS9, but actually fragment detected is | | | | | | | | | | |
| in 3′ UTR of PBEF, and 129050 encodes end of the | | | | | | | | | | |
| protein) | | | | | | | | | | |
| IL1B: Human interleukin 1-beta | gbh_m15330 | 47 | — | — | — | — | −3 | 2.4 | — | — |
| TNFAIP2: Homo sapiens tumor necrosis factor alpha- | gbh_m92357 | 48 | −1.6 | −6.3 | — | −3.7 | −7.1 | 3.1 | −2.2 | — |
| induced protein 2 | | | | | | | | | | |
| 02.01.04 Chemokines | | | | | | | | | | |
| H174. Homo sapiens putative alpha chemokine (H174) | gbh_af002985 | 49 | −3.5 | — | — | — | — | — | — | — |
| mRNA, complete cds. | | | | | | | | | | |
| GRO2 OR GROB OR MIP2A: Homo sapiens | gbh_af043340 | 50 | — | — | — | — | −5.6 | — | — | — |
| macrophage inflammatory protein-2-alpha | | | | | | | | | | |
| MIP-1A OR SCYA3 OR LD78: Homo sapiens mRNA | gbh_d00044 | 51 | −2.1 | — | — | — | −4.2 | — | — | — |
| for pLD78 peptide, complete cds. | | | | | | | | | | |
| SCYA5: Human small inducible cytokine A5 (aka T- | gbh_m21121 | 52 | −6.2 | — | — | — | 2.4 | — | — | — |
| cell specific RANTES protein | | | | | | | | | | |
| MONAP: Human monocyte-derived neutrophil- | gbh_m26383 | 53 | — | — | — | — | −24 | 8.6 | 8.5 | — |
| activating protein (MONAP) mRNA, also known as | | | | | | | | | | |
| Interleukin-8, or neutrophil-activating peptide-1. | | | | | | | | | | |
| IL8: Human interleukin 8 (IL8) gene, complete cds. | gbh_m28130 | 54 | −2.1 | — | — | — | −11 | 3.8 | 3.1 | — |
| Also known as neutrophil activating peptide-1, | | | | | | | | | | |
| monocyte derived neutrophil chemotactic factor, | | | | | | | | | | |
| granulocyte chemotactic protein 1, and emoctakin | | | | | | | | | | |
| SCYA4 OR MIP1B OR LAG1: Human small inducible | gbh_x16166 | 55 | — | — | — | — | — | — | 5.8 | — |
| cytokine A4 (aka MIP-1-beta, HC21, LAG-1) | | | | | | | | | | |
| TRK-T3: H. sapiens TRK-T3 oncogene | gbh_x85960 | 56 | — | −3.4 | −2.5 | −2.4 | −7.9 | 2.9 | — | — |
| 02.02.02 G-Protein Coupled Receptors | | | | | | | | | | |
| PTGER: Homo sapiens prostaglandin E2 receptor | gbh_125124 | 57 | — | — | — | — | −8.9 | — | — | — |
| 02.04.01 Alpha Subunits | | | | | | | | | | |
| GNAI3: Human guanine nucleotide-binding protein | gbh_j03005 | 58 | — | −3.3 | — | −2.4 | −3.8 | 4 | — | −3.9 |
| G(k), alpha subunit | | | | | | | | | | |
| 02.09 GTPase Activating Proteins | | | | | | | | | | |
| RGS1 OR 1R20 OR BL34: Human regulator of G- | gbh_s59049 | 59 | — | — | — | — | — | — | 6 | — |
| protein signaling (aka B-cell activation protein BL34) | | | | | | | | | | |
| 02.10.01 Stimulators | | | | | | | | | | |
| RAP1GDS1: Human RAP1 GTPase-GDP dissociation | hg2036ht2090 | 60 | — | — | — | −3.5 | −2.4 | 2.4 | — | — |
| stimulator 1 (aka smg P21 stimulatory GDP/GTP | | | | | | | | | | |
| exchange protein) | | | | | | | | | | |
| 02.11.01 Serine/Threonine Kinases | | | | | | | | | | |
| Human protein kinase C, iota type | gbh_118964 | 61 | — | −2.3 | — | −1.7 | −6 | 2.9 | — | — |
| : Homo sapiens casein kinase I, alpha isoform | gbh_137043 | 63 | — | −2.1 | −2 | −17 | −3.8 | 3.1 | 2.6 | — |
| : Human beta-adrenergic receptor kinase 1 (aka G- | gbh_m80777 | 64 | — | — | 4.6 | 5.7 | 3.6 | 10.1 | — | 3.2 |
| protein coupled receptor kinase 2) | | | | | | | | | | |
| : Homo sapiens protein phosphatase 1, regulatory | gbh_y13248 | 65 | — | −1.3 | — | −2.3 | −12 | 2.3 | 3.2 | — |
| subunit 10 | | | | | | | | | | |
| 02.12.02 Tyrosine Phosphatases | | | | | | | | | | |
| : Human HPTP epsilon mRNA for protein tyrosine | gbh_x54135 | 66 | — | — | — | — | −5.7 | 2.5 | — | — |
| phosphatase epsilon. | | | | | | | | | | |
| 02.12.03 Dual Specificity Phosphatases | | | | | | | | | | |
| : Homo sapiens MKP-1 like protein tyrosine | af038845 | 67 | — | — | — | — | — | — | 3.1 | — |
| phosphatase mRNA, complete cds - Homo sapiens, | | | | | | | | | | |
| 1471 bp (RNA). | | | | | | | | | | |
| : H. sapiens dual specificity protein phosphatase 6 | gbh_x93921 | 68 | — | 13 | — | 4.2 | — | — | 6.1 | — |
| 02.14 DNA Binding Proteins | | | | | | | | | | |
| : identical to b. 6165-6277 of AP000057 Homo sapiens | cghsr011113 5 | 69 | — | 3.3 | — | 3.5 | — | — | — | — |
| genomic DNA; ACUTE MYELOID LEUKEMIA 1 | _2 | | | | | | | | | |
| PROTEIN | | | | | | | | | | |
| : EST same as human snail protein homolog (aka zinc | gbeh_aa4650 | 69 | −3.9 | — | — | −4.1 | −9.5 | 10.1 | — | — |
| finger protein SNAH) | 53 | | | | | | | | | |
| : Human early growth response 2 | gbh_j04077 | 71 | — | 7.3 | — | 2.8 | 3 2 | 2.4 | 2.2 | — |
| : Human cyclic AMP-dependent transcription factor | gbh_m86843 | 72 | — | — | — | — | — | — | — | −1 2 |
| ATF-2 | | | | | | | | | | |
| 02.14.01 Transcription Factors | | | | | | | | | | |
| OTX1: Human homeobox protein OTX1 | cgp0t0hs175_ | 73 | 12.5 | 13.6 | 13.6 | 19.9 | 5.9 | −9.2 | — | — |
| | 3 | | | | | | | | | |
| ZINC FINGER PROTEIN PZF | gbeh_aa4213 | 74 | — | 1 5 | — | — | −3 | 2.3 | — | — |
| | 92 | | | | | | | | | |
| FOS | gbh_k00650 | 75 | — | 10.9 | — | 4 | — | −2.7 | — | — |
| STAT4 | gbh_178440 | 76 | — | −3 | −2.2 | −2.1 | −2.5 | 3 | — | −9.8 |
| RARG | gbh_m38258 | 77 | — | −4.9 | — | — | — | — | — | — |
| : Human retinoic acid receptor gamma 1 | gbh_m38259 | 78 | — | −3.9 | — | — | — | — | — | — |
| : Human ETS-related transcription factor (aka E74-like | gbh_m82883 | 79 | — | — | — | — | −7.3 | 9 | — | 2.3 |
| factor 1) | | | | | | | | | | |
| NRF2 | gbh_s74017 | 78 | — | — | — | — | −5.4 | 2.4 | — | — |
| TAF2F | gbh_u18062 | 79 | — | — | — | — | −4.5 | 5.3 | 2.4 | — |
| ZNF15 | gbh_u22377 | 80 | — | −2.1 | −2 | −2.4 | −23 | 8.8 | 2.1 | — |
| FOS | gbh_v01512 | 81 | — | 6.5 | — | 2.4 | — | −3.9 | — | — |
| FOSB | hg2253ht2338 | 82 | −2.1 | 10.5 | — | 2.5 | −2.8 | 10.4 | — | — |
| FOS | uehsf_17878_0 | 83 | — | 7.6 | — | 3.8 | — | −2.7 | — | — |
| 02.14.04 Transcription Repressors | | | | | | | | | | |
| Human cAMP-responsive element modulator alpha | gbh_s68271 | 84 | — | — | — | — | — | — | 3.9 | — |
| isoform | | | | | | | | | | |
| 03.01.02 Chromatin Structure | | | | | | | | | | |
| : Human H3.3 histone class C | gbh_m11353 | 85 | — | −3 | — | −5.2 | — | 14.8 | — | — |
| 03.02.01 Polymerases | | | | | | | | | | |
| : Human DNA polymerase delta catalytic chain | gbh_m80397 | 86 | — | — | — | — | −2.8 | — | −3.2 | — |
| 03.03.02 Cyclins | | | | | | | | | | |
| : H. sapiens G2/mitotic-specific cyclin F | gbh_z36714 | 87 | — | — | — | — | — | −4 | 4.1 | — |
| 03.03.04 Kinetochore Interface | | | | | | | | | | |
| : Human G2/mitotic-specific cyclin A2 | gbh_x51688 | 88 | 2 2 | 2.1 | 1.8 | 2.5 | −3.3 | 8.4 | — | — |
| 03.03.05 G0/G1 Switch Genes | | | | | | | | | | |
| : Human helix-loop-helix basic phosphoprotein (G0S8) | gbh_113463 | 89 | — | 2.1 | — | — | −2.2 | −3 5 | 5.7 | — |
| mRNA, complete | | | | | | | | | | |
| 03.03.06.02 Apoptosis Inhibition | | | | | | | | | | |
| : Human tumor necrosis factor alpha inducible protein | gbh_m59465 | 90 | — | — | — | −2.4 | −5 | 10 7 | — | — |
| 3 (zinc finger protein A20) | | | | | | | | | | |
| 04.02.01 Cholesterol Biosynthesis | | | | | | | | | | |
| : Homo sapiens squalene monooxygenase (aka | gbh_d78130 | 91 | — | −23 | — | −2.2 | −5.1 | 9 6 | — | 1 9 |
| squalene epoxidase) | | | | | | | | | | |
| : Human cytochrome P450 51 (aka lanosterol 14- | gbh_u23942 | 92 | −2.6 | −3.3 | −1.5 | −5.9 | −4.5 | 5.8 | — | — |
| demethylase) | | | | | | | | | | |
| : Human cytochrome P450 51 (aka lanosterol 14-alpha- | gbh_u36926 | 93 | −2.3 | −3 | — | — | −13 | 9.3 | — | — |
| demethylase) | | | | | | | | | | |
| : Human C-4 methyl sterol oxidase | gbh_u60205 | 94 | — | −4.2 | −2.1 | −3.7 | −5.5 | 2.6 | — | 1.6 |
| 04.02.02 Steroid Hormone Biosynthesis | | | | | | | | | | |
| : zm68c06.rl Stratagene neuroepithelium (#937231), | gbeh_aa070298 | 95 | — | — | — | — | −15 | 8.3 | 1.7 | — |
| similar to mouse PUTATIVE STEROID | | | | | | | | | | |
| DEHYDROGENASE KIK-1 [C] | | | | | | | | | | |
| 04.03.02 Glycogen Manipulation | | | | | | | | | | |
| : Homo sapiens 1,4-alpha-glucan branching enzyme | gbh_107956 | 96 | — | — | — | — | −6.7 | 3.4 | 2.2 | 1.7 |
| 04.03.05 Pentose Phosphate Pathway | | | | | | | | | | |
| : Homo sapiens transaldolase | gbh_119437 | 97 | — | — | — | — | — | — | — | 4.5 |
| : H. sapiens transketolase | gbh_x67688 | 98 | — | — | — | — | — | — | — | −2 |
| 04.04 Oxidative Phosphorylation | | | | | | | | | | |
| : Human mitochondrial NADH dehydrogenase subunit | 100016 | 99 | — | — | 95.2 | — | 100 | −23.1 | — | — |
| 4 (complex I); aka NADH-UBIQUINONE | | | | | | | | | | |
| OXIDOREDUCTASE, SUBUNIT ND4 | | | | | | | | | | |
| 04.04.03 ATP/Proton Motive Force | | | | | | | | | | |
| Interconversion | | | | | | | | | | |
| : H. sapiens ATP synthase alpha chain, mitochondrial | gbh_x65460 | 100 | 3.4 | 3.3 | 3.2 | 3.1 | 23 | −2 1 | −2.7 | — |
| 04.08.01 Polyamine Metabolism | | | | | | | | | | |
| SAT or ATDA | | | | | | | | | | |
| : Human diamine acetyltransferase; | gbh_m55580 | 101 | — | −3 | −2.2 | — | −5.2 | 9.2 | — | — |
| spermidine/spermine N1-acetyltransferase (SSAT); | | | | | | | | | | |
| Putrescine acetyltransferase | | | | | | | | | | |
| 04.11.01.02 Lipids | | | | | | | | | | |
| LDLR | | | | | | | | | | |
| : Human low density lipoprotein receptor | gbh_129401 | 102 | −2.1 | −3.1 | −2.2 | −2.8 | −3.5 | 3.5 | 2.2 | — |
| ADRP | | | | | | | | | | |
| : H. sapiens adipose ifferentiation- related protein (aka | gbh_x97324 | 103 | −2.3 | −3.2 | −2.1 | −3.9 | −12 | 14.9 | 3.7 | — |
| adipophilin) | | | | | | | | | | |
| 05.01 Cytoskeleton | | | | | | | | | | |
| : AF077046 Homo sapiens ganglioside expression | cghsg0c0l79. | 104 | 4.6 | 2.2 | 2 | 2.1 | — | — | — | — |
| factor | 2_3 | | | | | | | | | |
| 05.01.01 Components | | | | | | | | | | |
| : H. sapiens beta tubulin | gbh_x79535 | 105 | — | — | — | — | −1 5 | 1.4 | 2.5 | — |
| 05.02.02 ECM Breakdown | | | | | | | | | | |
| : Human plasminogen (contains angiostatin) | gbh_m74220 | 106 | — | −2.3 | — | −2 | −3.4 | 3.8 | — | — |
| 05.02.03 ECM Breakdown Inhibition | | | | | | | | | | |
| : Human metalloproteinase inhibitor 3 [C] | gbh_u38955 | 107 | — | — | — | — | −10 | 4.4 | — | — |
| 05.03 Intercellular Adhesion | | | | | | | | | | |
| : Human platelet endothelial cell adhesion molecule | gbh_m28526 | 108 | — | — | — | — | −5.6 | — | — | — |
| (PECAM-1) mRNA, | | | | | | | | | | |
| 05.03.02 Interface with Extracellular Matrix | | | | | | | | | | |
| : Human cell surface adhesion glycoproteins LFA- | gbh_m15395 | 109 | — | −2.2 | — | −1.5 | −2.2 | 3.3 | — | — |
| 1/CR3/P150,95 beta-subunit (aka integrin beta-2, | | | | | | | | | | |
| CD18) | | | | | | | | | | |
| 06.01.01 Nucleotidases | | | | | | | | | | |
| : EST assembly same as human 5′ nucleotidase (aka | uehsf_8520_0 | 110 | — | — | — | — | — | — | 10.9 | — |
| ecto-nucleotidase, CD73 antigen) | | | | | | | | | | |
| 06.01.02 Peptidases | | | | | | | | | | |
| : Human cathepsin S [C] | gbh_m90696 | 111 | −2.3 | −2.5 | −2 | −3.8 | −4.3 | 3.3 | — | — |
| 07.02.03 Ion Pumps | | | | | | | | | | |
| : Homo sapiens calcium-transporting ATPase | gbh_m23114 | 112 | — | — | 2.5 | −2.3 | — | 6.3 | — | — |
| sarcoplasmic reticulum type, class 2 isoform (aka | | | | | | | | | | |
| HK1) | | | | | | | | | | |
| 07.04 Peroxisome/Lysosome/Endosome | | | | | | | | | | |
| : Homo sapiens palmitoyl-protein thioesterase | gbh_142809 | 113 | — | — | — | — | −4.9 | 3.5 | — | — |
| 08.03 Vesicle Trafficking | | | | | | | | | | |
| Beige (CHS) or LYST | | | | | | | | | | |
| : Human beige protein homolog (chs) mRNA, | gbh_u67615 | 114 | −2 | −4.2 | −1.8 | −2.3 | −5.1 | 3.9 | 3.2 | — |
| complete cds; Human lysosomal trafficking regulator | | | | | | | | | | |
| (LYST) | | | | | | | | | | |
| 09 Unknown Function | | | | | | | | | | |
| : same as patented sequence V08824 | gbeh_aa082569 | 115 | — | — | — | — | −3.4 | — | — | — |
| : zq40h11s1 Stratagene hNT neuron (#937233) Homo | gbeh_aa160975 | 116 | — | — | — | — | −8 | 4 | — | — |
| sapiens cDNA clone 632229 3″ similar to WP: | | | | | | | | | | |
| T09F3.2 CE02342 CARRIER PROTEIN C2 [N] | | | | | | | | | | |
| : EST177650 Jurkat T-cells VI Homo sapiens cDNA 5″ | gbeh_aa306714 | 117 | — | — | — | — | −5.9 | — | — | — |
| end [N] | | | | | | | | | | |
| : EST178616 Colon carcinoma (HCC) cell line Homo | gbeh_aa307755 | 118 | — | — | — | — | — | 3.1 | 4 2 | — |
| sapiens cDNA 5″ end [N] | | | | | | | | | | |
| : EST187439 Colon carcinoma (HCC) cell line II | gbeh_aa315546 | 119 | — | — | — | −4.8 | — | — | — | — |
| Homo sapiens cDNA 5″ end | | | | | | | | | | |
| : EST72341 Namalwa B cells I Homo sapiens cDNA | gbeh_aa362484 | 120 | — | — | — | — | −3.2 | — | — | — |
| 3″ end [N] | | | | | | | | | | |
| : zx46a02.rl Soares testis NHT Homo sapiens cDNA | gbeh_aa451786 | 122 | −6.9 | −1.5 | −1.4 | −1.7 | −3.2 | 2 | — | — |
| clone 795242 5″ | | | | | | | | | | |
| gheh_aa486012 | | | | | | | | | | |
| : ab38d11.r1 Stratagene HeLa cell s3 937216 Homo | gbeh_aa486012 | 123 | — | — | — | — | — | — | — | −5.3 |
| sapiens cDNA clone 843093 5″ | | | | | | | | | | |
| : no14b05.s1 NCI_CGAP_Phel Homo sapiens cDNA | gbeh_aa601191 | 124 | — | 4.4 | — | — | — | −9.8 | — | — |
| clone IMAGE: 1100625 3″ | | | | | | | | | | |
| : ag85h03.r1 Stratagene hNT neuron (#937233) Homo | gbeh_aa722311 | 125 | 3 | 6.8 | 2.6 | — | — | — | — | — |
| sapiens cDNA clone [N] | | | | | | | | | | |
| : oh38d12.s1 NCI_CGAP_Kid6 Homo sapiens cDNA | gbeh_aa864847 | 126 | — | — | — | — | −3.4 | — | — | — |
| clone IMAGE:1460087 3″ similar to contains PTR5 t3 | | | | | | | | | | |
| PTR5 repetitive element [N] | | | | | | | | | | |
| : yu87b03 r1 Homo sapiens cDNA clone 240749 5″ | gbeh_h91343 | 127 | −2.1 | −3.1 | — | −5.6 | −4 8 | 3.9 | — | — |
| similar to contains Alu repetitive element [N] | | | | | | | | | | |
| : yb25f12.s1 Homo sapiens cDNA clone 72239 3″ | gbeh_t51565 | 128 | — | — | — | — | 7.3 | — | — | — |
| contains MSR1 repetitive element [N] | | | | | | | | | | |
| : Homo sapiens SS-A/Ro autoantigen 52 kda | gbh_u01882 | 129 | — | — | — | — | — | — | — | −4.3 |
| component gene, complete | | | | | | | | | | |
| : ys68b01 s1 Homo sapiens cDNA, 3″ end | uehsf_27449_1 | 130 | −3.7 | — | −1.8 | — | — | — | — | 1.9 |
| : yg40f03.r1 Homo sapiens cDNA, 5″ end | uehsf_53740_0 | 131 | — | — | — | — | — | — | — | −3.9 |
| : yp92d11.r1 Homo sapiens cDNA, 5″ end | uehsf_53874_0 | 132 | — | — | — | — | — | — | 3.7 | 2.9 |
| 09.01 Known Genes | | | | | | | | | | |
| : Homo sapiens growth-arrest-specific protein (gas) | gbh_113720 | 133 | — | — | 2.5 | — | — | — | — | — |
| mRNA, complete | | | | | | | | | | |
| 09.01.01 Disease-Associated | | | | | | | | | | |
| : Human breast cancer type 2 susceptibility protein | gbh_u43746 | 134 | — | −3 | — | −5.2 | −9.7 | 14.8 | — | — |
| 09.01.01 01 Cancer | | | | | | | | | | |
| : Human Int-6 | gbh_u62962 | 135 | — | — | — | — | −2.5 | 2.4 | — | 4.5 |
| 09.01.02 Unassociated | | | | | | | | | | |
| : Homo sapiens mRNA for KIAA0618 protein, | gbh_ab01451 | 136 | 3.6 | 2.4 | 2.8 | — | −1.5 | −2.9 | — | — |
| complete cds. | | | | | | | | | | |
| : Homo sapiens mRNA for KIAA0768 protein, partial | gbh_ab01831 | 137 | — | 2.1 | — | — | −3.6 | −2.3 | — | — |
| cds. | | | | | | | | | | |
| : Human hypothetical protein KIAA0274 | gbh_d87464 | 138 | — | — | — | — | — | — | — | −2 |
| 09.02 Putative Homologies | | | | | | | | | | |
| : zc23g11.r1 Soares senescent fibroblasts NbHSF | gbeh_w45240 | 139 | — | −2.1 | — | — | −2.5 | 2.1 | 2.1 | −2.2 |
| Homo sapiens cDNA clone 323204 5″ similar to | | | | | | | | | | |
| gb:M54915 PIM-1 PROTO-ONCOGENE | | | | | | | | | | |
| SERINE/THREONINE-PROTEIN KINASE | | | | | | | | | | |
| (HUMAN) [N] | | | | | | | | | | |

Key to treatment groups:
4798 = collagen type I
4799 VACM
4800 = laminin
4801 = fibronectin
4802 = fibronectin
4865 = poly lysine
5552 = VCAM
5553 = fibronectin
6892 = VCAM
5554 = polylysine
6551 = fibronectin
6552 = VCAM
6554 = polylysine
6556 = fibronectin
6890 = fibronectin
6891 = VCAM

TABLE 3
Known Genes

| | GenBank | KEANO | | | | | | | | |
|---|---|---|---|---|---|---|---|---|---|---|
| Description of Sequences | Acc. No. | X | 5554 | 6551 | 6552 | 6554 | 6556 | 6890 | 6891 | 6892 |
| 00 Unassigned | | | | | | | | | | |
| gbh af043143 | gbh_af043143 | 140 | — | — | 2.3 | — | 2 3 | — | — | 2 5 |
| : Homo sapiens interleukin-1 intracellular receptor | | | | | | | | | | |
| antagonist | | | | | | | | | | |
| gbh 176191 | gbh_176191 | 141 | — | — | −2 1 | 2.5 | 2.1 | 3.6 | — | −2.2 |
| : Homo sapiens interleukin-1 receptor- | | | | | | | | | | |
| associated kinase (IRAK) mRNA, | | | | | | | | | | |
| gbh m83221 | gbh_m83221 | 142 | — | — | — | — | 3.8 | — | — | — |
| : Homo sapiens I-Rel mRNA, complete cds | | | | | | | | | | |
| gbh u91616 | gbh_u91616 | 143 | — | 2.5 | — | 2.8 | 3.5 | — | — | 2.9 |
| : Human I kappa B epsilon (IkBe) mRNA, complete | | | | | | | | | | |
| cds | | | | | | | | | | |
| 01.01.01 Polymerases | | | | | | | | | | |
| OASL or TRIP14 | gbh_aj225089 | 144 | 2.3 | 3 | −3.5 | 3.2 | 8 2 | −3.1 | −4.6 | — |
| : Homo sapiens 59 kDa 2′-5′ oligoadenylate | | | | | | | | | | |
| synthetase-like | | | | | | | | | | |
| protein (aka thyroid receptor interacting protein 14) | | | | | | | | | | |
| 01.01.03 mRNA Splicing | | | | | | | | | | |
| PSF | gbh_x70944 | 145 | — | 2.8 | 5.9 | — | 2.3 | −2 2 | −41 | 2 1 |
| : H. sapiens mRNA for PTB-associated splicing factor. | | | | | | | | | | |
| 01.01.04 Regulators | | | | | | | | | | |
| IRF1 | gbh_105072 | 146 | — | 2.6 | 2 7 | −5.4 | 2.3 | −3 | — | — |
| : Homo sapiens interferon regulatory factor 1 | | | | | | | | | | |
| BCL3 | gbh_m31732 | 142 | 2.1 | 3 6 | — | 5.5 | 11 3 | 6 4 | 6.7 | — |
| : Human B-cell lymphoma 3-encoded protein | | | | | | | | | | |
| 01.02.01 Ribosomal Proteins | | | | | | | | | | |
| RPS20 | gbh_106498 | 148 | — | — | — | 6 8 | 10 5 | — | −2.1 | — |
| : Homo sapiens ribosomat protein S20 mRNA, | | | | | | | | | | |
| complete cds. | | | | | | | | | | |
| RPL37A | gbh_106499 | 149 | — | — | — | — | — | −18.8 | −24.2 | — |
| : Homo sapiens 60S ribosomal protein L37a | | | | | | | | | | |
| RPL8 | gbh_z28407 | 150 | — | — | — | — | 2.2 | −11.1 | −7.5 | — |
| H. sapiens 60S ribosomal protein L8 | | | | | | | | | | |
| 01.02.04 Nucleoproteins | | | | | | | | | | |
| HNRPHI or HNRPH | gbh_122009 | 151 | — | — | 2.6 | — | — | 5.3 | — | — |
| : Human heterogeneous nuclear ribonuclearprotein H | | | | | | | | | | |
| 01.02.05 Translation Factors | | | | | | | | | | |
| EF2 | gbh_m19997 | 152 | −3 8 | 3 8 | 3.7 | −3.7 | — | 10 6 | 5 7 | −5.9 |
| : Human elongation factor 2 (EF-2) mRNA, 3′ end. | | | | | | | | | | |
| 01.02.06 Ribosomal RNA's | | | | | | | | | | |
| 28s rRNA | m11167 | 153 | — | — | 5.2 | — | — | — | 4 | — |
| : Human 28S ribosomal RNA gene. | | | | | | | | | | |
| 18S rRNA | x03205 | 154 | — | −3.1 | — | — | — | — | — | — |
| : Human 18S ribosomal RNA-Homo sapiens, | | | | | | | | | | |
| 1869 bp. | | | | | | | | | | |
| 01.03.01 Molecular Chaperone | | | | | | | | | | |
| Cyclophilin/PPIASE | ai471997 | 155 | — | — | — | 4 | 4 4 | — | — | 7 2 |
| p60 EBI3-assoc protein | gbh_u46751 | 156 | — | 2 5 | 3 | 10 9 | 9 9 | −4.7 | −3 8 | 2 2 |
| CAPN4 or CAPNS | gbh_x04106 | 157 | — | 2.3 | 2 1 | 2.7 | 3.3 | 2 | 2.8 | — |
| 01.05.02 Ubiquination | | | | | | | | | | |
| UCBH8 | gbh_af031141 | 158 | — | — | −6.7 | 6.1 | 1.5 | — | — | — |
| G1P2 or UCRP or ISG15 | hg412ht412 | 159 | 2 7 | — | −3.6 | 8 6 | — | — | — | 9 |
| G1P2 or UCRP or ISG15 | uehsf_41281 | 160 | 2.7 | — | −3.6 | 13.4 | 14 8 | — | — | — |
| 02 Signal Transduction | | | | | | | | | | |
| IF141 | gbh_122342 | 161 | — | — | — | — | 4 7 | — | — | — |
| : Human interferon-induced protein 41, 30 kDa | | | | | | | | | | |
| 02.01.02 Growth Factors | | | | | | | | | | |
| P17 or PN1 | gbph_ao391 | 162 | — | 2 7 | 2.9 | — | 1 6 | — | — | 16 4 |
| : H. sapiens glia derived nexin (aka glia derived | | | | | | | | | | |
| neurite promoting factor, protease nexin I, protease | | | | | | | | | | |
| inhibitor 7) | | | | | | | | | | |
| 02.01.03 Cytokines | | | | | | | | | | |
| IL-B30 | gbeh_aa4189 | 163 | — | 2.7 | 2 | 6 6 | 27.7 | — | — | 8.3 |
| : zw01c10.rl Soares NhHMPu S1 Homo sapiens cDNA | | | | | | | | | | |
| clone 768018 5″ | | | | | | | | | | |
| IL1B | gbh_k02770 | 164 | — | — | — | 6 7 | 8.4 | — | — | — |
| : Human interleukin-1 beta | | | | | | | | | | |
| TNF | gbh_m10988 | 165 | — | 6.4 | — | — | 28 4 | — | — | — |
| : Human tumor necrosis factor | | | | | | | | | | |
| ILlB | gbh_m15330 | 166 | — | — | — | 6.7 | 5.7 | — | — | — |
| : Human interleukin 1-beta | | | | | | | | | | |
| TNFAIP2 | gbh_m92357 | 167 | — | 8.7 | — | 16.9 | 16.7 | — | 6 9 | 2.4 |
| : Homo sapiens tumor necrosis factor alpha-induced | | | | | | | | | | |
| protein 2 | | | | | | | | | | |
| PBEF | gbh_u02020 | 166 | — | 3.3 | 3.7 | 17.2 | 14.3 | 2 9 | — | 4.4 |
| : Human pre-B cell enhancing factor (PBEF) mRNA, | | | | | | | | | | |
| complete cds. | | | | | | | | | | |
| TNF | gbh_x01394 | 169 | — | — | — | — | 26 4 | — | — | — |
| : Human mRNA for tumor necrosis factor. | | | | | | | | | | |
| 02.01.04 Chemokines | | | | | | | | | | |
| MIP-1A or SCYA3 or LD78 | gbh_d00044 | 170 | — | −2.1 | −2 7 | 10.4 | 12.4 | 4.9 | — | — |
| : human monocyte chemoattractant protein-1 | | | | | | | | | | |
| INP10 | ghh_x02530 | 172 | — | — | −23 | 10.5 | 11.1 | — | — | 11.5 |
| : Human interferon-gamma induced protein | | | | | | | | | | |
| INP10 | hg1192ht1192 | 173 | — | — | −9 | 8 3 | 11.1 | — | — | 11.5 |
| : monocyte chemotactic protein 1; monocyte | | | | | | | | | | |
| chemotactic and activating factor; monocyte secretory | | | | | | | | | | |
| protein JE (HC1 1) | | | | | | | | | | |
| MIP-1A or SCYA3 or LD78 | i68195 | 175 | — | — | — | 6.7 | 6 8 | — | — | — |
| 02.01.05 Peptide Hormone Binding Proteins | | | | | | | | | | |
| IGFBP3 or IBP3 | gbh_m31159 | 176 | — | — | — | 37 | 2.6 | — | — | 16.1 |
| : Human insulin-like growth factor binding protein 3 | | | | | | | | | | |
| 02.02 Peptide Receptors | | | | | | | | | | |
| IL10R or IL10RA | gbh_u00672 | 177 | 3.5 | 6.3 | −2 7 | 19 | 13.2 | — | — | 3 |
| : Human interleukin-10 receptor | | | | | | | | | | |
| TNFRSF5 or CD40 | gbh_x60592 | 178 | 1.8 | — | — | 6 3 | 9 6 | — | — | — |
| EB13 | uehaf_1479_2 | 179 | — | 2.5 | — | 25 6 | 32.7 | — | 3.1 | — |
| 02.02.01 Tyrosine Kinase Receptors | | | | | | | | | | |
| ENG or END | gbh_x72012 | 180 | — | 4 | 5 3 | 2.2 | 1 7 | — | — | −2 3 |
| - activating transcription factor 3 (ATF3)(L19871), TSt | | 181 | 3.8 | — | — | 5.2 | 2.4 | — | — | −2.2 |
| yj50h01.rl | | | | | | | | | | |
| 03.03.05 G0/G1 Switch Genes | | | | | | | | | | |
| G0S2 | gbh_m69199 | 182 | — | — | — | 9.9 | 7 7 | — | — | — |
| : Human putative lymphocyte G01/G1 switch protein 2 | | | | | | | | | | |
| 03.03.06.02 Apoptosis Inhibition | | | | | | | | | | |
| TNFAIP3 | gbh_m59465 | 183 | — | 2.9 | — | 3.3 | 3.1 | — | — | 2 2 |
| : Human tumor necrosis factor alpha inducible | | | | | | | | | | |
| protein 3 (zinc finger protein A20) | | | | | | | | | | |
| 04.01.01 Fatty Acid Synthesis | | | | | | | | | | |
| LACS1 or FACL1 | ghh_d10040 | 184 | 2 7 | — | 2.5 | 9.1 | 11 5 | — | — | 6 6 |
| : Homo sapiens long-chain acyl-CoA synthetase 1 (aka | | | | | | | | | | |
| long-chain-fatty-acid-coA ligase 1) | | | | | | | | | | |
| :Homo sapiens dihydroxyacetone phosphate | | 185 | | | | | | | | |
| acyltransferase | | | | | | | | | | |
| 04.02.01 Cholesterol Biosynthesis | | | | | | | | | | |
| FDFT1 | gbh_x69141 | 186 | — | — | — | −3.5 | −2 7 | — | — | 5 3 |
| : H. sapiens mRNA for squslene synthase | | | | | | | | | | |
| 04.03.05 Pentose Phosphate Pathway | | | | | | | | | | |
| hPGDH | gbh_u16900 | 187 | — | — | — | 2.1 | 4 1 | — | — | — |
| : H. sapiens carbonic anhydrase II | | 188 | | | | | | | | |
| 04.05.03 Glutamate Family | | | | | | | | | | |
| GLCL | uehsf_169000 | 189 | — | −2 4 | — | 10 2 | 2 9 | — | 9 8 | 2 6 |
| : Human glutamate-cysteine ligate catalytic subunit | | | | | | | | | | |
| 04.07 Biosynthesis of Cofactors, | | | | | | | | | | |
| Prosthetic Groups, Carriers | | | | | | | | | | |
| kynureninase | gbh_u51721 | 190 | — | — | — | 3 7 | 3 | — | — | 5.2 |
| : Human kynureninase (aka L-kynurenine hydrolase) | | | | | | | | | | |
| 04.08.01 Polyamine Metabolism | | | | | | | | | | |
| SAT or ATDA | gbh_m77693 | 191 | — | — | — | 10.4 | 8.7 | — | — | — |
| : Human diamine acetyltransferase; spermidine/ | | | | | | | | | | |
| spermine N1-acetyltranaferase (SSAT), Putrescine | | | | | | | | | | |
| acetyltransferase | | | | | | | | | | |
| 04.09.02 Oxygen Radicals | | | | | | | | | | |
| SOD2 | gbh_x65965 | 192 | — | 2.1 | — | 4.6 | 9 7 | — | — | — |
| : H. sapiens manganese superoxide diamutase | | | | | | | | | | |
| SOD2 | uehsf_737_2 | 193 | — | — | — | 39.4 | 32.4 | 2 1 | — | — |
| 04.09.03 Heavy Metals | | | | | | | | | | |
| MT2 or MT2A | gbh_j00272 | 194 | — | — | — | 11 8 | 19.6 | — | — | — |
| : Human metallothionein-II pseudogene | | | | | | | | | | |
| 04.11.01.02 Lipids | | | | | | | | | | |
| ADRP | gbh_x97324 | 195 | — | — | — | 3.5 | 4.5 | — | — | −5.7 |
| : H.sapiens adipose ifferentiation-related protein | | | | | | | | | | |
| (aka adipophilin) | | | | | | | | | | |
| : Human low density lipoprotein receptor | | 196 | | | | | | | | |
| 04.11.03.02 Iron | | | | | | | | | | |
| FTH1 or FTH or FTHL6 | gbh_120941 | 197 | — | 2 4 | — | 3 9 | 8.4 | — | — | 2.8 |
| : Human ferritin heavy chain (aka ferritin H subunit) | | | | | | | | | | |
| 05.01.01.02 Structural Arm: Intermediate Filaments | | | | | | | | | | |
| LMNI or LMNA | gbh_m13451 | 198 | — | 5 2 | 2.9 | — | 2 5 | — | — | — |
| : Human lamin C | | | | | | | | | | |
| 05.01.01.03 Structural Arm: Actins & Short Filaments | | | | | | | | | | |
| ACTB | gbh_x00351 | 199 | −4.7 | 4.5 | 5 1 | −3.4 | — | 23 5 | — | — |
| : Homo sapiens WDRI protein mRNA, complete cds. | | 200 | | | | | | | | |
| 05.02 Extracellular Matrix | | | | | | | | | | |
| CHI3L1 | gbh_y08378 | 201 | — | 4.3 | — | — | 4 3 | −2.2 | −2 | — |
| : H. sapiens cartilage glycoprotein-39 (aka chitinase-3 | | | | | | | | | | |
| like 1) | | | | | | | | | | |
| 05.02.01 ECM Component | | | | | | | | | | |
| SDC2 or HSPG1 | gbh_j04621 | 202 | — | — | 2 4 | −2 3 | −1 7 | — | — | 3 6 |
| : Human syndecan-2 (aka heparan sulfate proteoglycan | | | | | | | | | | |
| core protein) | | | | | | | | | | |
| COL9A2 | gbh_m95610 | 203 | −2.9 | 8.1 | 3.6 | −6 6 | 1 9 | −8 2 | 5.4 | — |
| : Human collagen alpha 2(IX) chain | | | | | | | | | | |
| 05.03.02 Interface with Extracellular Matrix | | | | | | | | | | |
| ITGB2 or CD18 | gbh_m15395 | 204 | 2.2 | — | 10.3 | −5.2 | 2 6 | 19 8 | 17 2 | −2.7 |
| : Human cell surface adhesion glycoproteins LFA-1/ | | | | | | | | | | |
| CR3/P150,95 beta-subunit (aka integrin beta-2, CD18) | | | | | | | | | | |
| CD44 | gbh_m69215 | 205 | — | — | — | 9.8 | 18 7 | 2 9 | — | 2 |
| : Human CD44 antigen (aka hyaluronate receptor) | | | | | | | | | | |
| ITGB2 or CD18 | gbh_x6407110 | 206 | 2 2 | 2.5 | 10 3 | −5 2 | — | 19 8 | 17 2 | −2 7 |
| : H. sapiens CD18 exon 1 | | | | | | | | | | |
| ITGB2 or CD18 | gbh_x6407111 | 207 | 2.2 | 14 1 | 10.3 | −5.2 | — | 19 8 | 17 2 | −2.7 |
| 06.01.02 Peptidases | | | | | | | | | | |
| CTSC | gbh_u79415 | 208 | 2 4 | — | — | 4 8 | 12.8 | 13 1 | 6 7 | 10.3 |
| : Homo sapiens dipeptidyl-peptidase I (aka cathepsin | | | | | | | | | | |
| C/J, dipeptidyl transferase) | | | | | | | | | | |
| 06.02 Immune System | | | | | | | | | | |
| gbh x03557 | gbh_x03557 | 209 | — | — | −5 2 | 33.4 | 38 5 | 3 5 | — | — |
| : Human mRNA for 56-KDa protein induced by | | | | | | | | | | |
| interferon [C] | | | | | | | | | | |
| 06.02.02.01 MHC Proteins | | | | | | | | | | |
| TAP2E, PSF-2 | gbh_m84748 | 210 | — | 5 1 | 3.3 | −4.1 | 6 8 | — | 2.3 | 2 |
| : Human MHC class II gene. | | | | | | | | | | |
| 06.02.03 T-Cell Activation and Proliferation | | | | | | | | | | |
| E16/CD98/LAT1 | Gbh_8024 | 211 | 3 8 | 6.8 | 5.9 | −4 1 | 2 3 | −3 6 | — | — |
| : Human E16 MRNA, complete cds., ident. | | | | | | | | | | |
| CD98/LAT1. | | 212 | | | | | | | | |
| : Human R2 mRNA for an inducible membrane | | | | | | | | | | |
| protein, C33 ANTIGEN, 1A4, Metastasis Suppressor | | | | | | | | | | |
| Kangai I; Suppressor of Tumorigenicity-6 | | | | | | | | | | |
| 06.03.03 Platelet Function | | | | | | | | | | |
| PLEK or P47 | gbh_x07743 | 213 | — | — | 3.1 | 2 9 | 4 | — | — | 2 8 |
| : Human pleckstrin (aka platelet P47 protein) | | | | | | | | | | |
| 06.03.03.02 Aggregation Inhibition | | | | | | | | | | |
| ANX8 | gbh_x16662 | 214 | — | 3 | — | 4.1 | — | — | 4.1 | |
| : Human sodium/potassium-transporting ATPase | | | | | | | | | | |
| beta-1 chain | | | | | | | | | | |
| 07.04.01 H+ Regulation | | | | | | | | | | |
| ATP6N1 or VPP1 | gbh_u45285 | 216 | — | 4.8 | 4.5 | −4.2 | −2.6 | — | — | — |
| : Human clathrin-coated vesicle/synaptic vesicle proton | | | | | | | | | | |
| pump 116 kDa subunit (aka vacuolar proton | | | | | | | | | | |
| pump subunit 1) | | | | | | | | | | |
| 08.03.01 Early Endosome Formation/Fusion | | | | | | | | | | |
| VAMP8 | gbh_sf05323 | 217 | — | 4 2 | — | −10.3 | −1.8 | — | — | — |
| : Homo sapiens vesicle-associated membrane protein 8 | | | | | | | | | | |
| (aka endobrevin) | | | | | | | | | | |
| 09 Unknown Function | | | | | | | | | | |
| gbeh aa082841 | gbeh_aa082841 | 218 | — | — | 6.2 | — | — | — | — | — |
| : zn08a01.s1 Stratagene hNT neuron (#937233) Homo | | | | | | | | | | |
| sapiens cDNA clone 546792 3″ [C] [N] | | | | | | | | | | |
| gbeh aa134958 | gbeh_aa134958 | 219 | — | — | −2 9 | 11.7 | 11 7 | — | — | — |
| : zo23f04.s1 Stratagene colon (#937204) Homo | | | | | | | | | | |
| sapiens cDNA clone 587743 3″[N] | | | | | | | | | | |
| gbeh aa179194 | gbeh_aa179194 | 220 | — | — | — | — | — | — | — | −3.3 |
| : zp45e04.s1 Stratagene HeLa cell s3 937216 Homo | | | | | | | | | | |
| sapiens cDNA clone 612414 3″ similar to contains | | | | | | | | | | |
| Alu repetitive element | | | | | | | | | | |
| gbeh aa337945 | gbeh_aa337945 | 221 | — | — | — | 5.2 | 8 4 | −3 9 | — | — |
| : EST42747 Endornetrial tumor Homo sapiens | | | | | | | | | | |
| cDNA 5″ end [N] | | | | | | | | | | |
| gbeh aa365298 | gbeh_aa365298 | 222 | — | 1 7 | — | 3 | 5 3 | — | — | — |
| : EST76292 Pineal gland II Homo sapiens | | | | | | | | | | |
| cDNA 3″ end [N] | | | | | | | | | | |
| gbeh aa385535 | gbeh_aa385535 | 223 | 2 6 | — | — | 14.4 | — | — | — | — |
| : EST99198 Thyroid Homo sapiens cDNA 5″ end [N] | | | | | | | | | | |
| gbeh aa524525 | gbeh_aa524525 | 224 | — | — | — | — | — | — | — | −2 |
| : ng44h03 s1 NC1_CGAP_Co3 Homo sapiens cDNA | | | | | | | | | | |
| clone IMAGE.937685 | | | | | | | | | | |
| gbeh aa586814 | gbeh_aa586814 | 225 | — | — | 2 1 | 2.5 | 4 6 | — | — | — |
| : nn79b06.s1 NCI_CGAP_Co9 Homo sapiens cDNA | | | | | | | | | | |
| clone IMAGE·1090067 3″, [N] | | | | | | | | | | |
| gbeh aa853401 | gbeh_aa853401 | 226 | 14.3 | — | — | — | — | — | — | — |
| : NHTBCae05e03r1 Normal Human Trabecular Bone | | | | | | | | | | |
| Cells Homo sapiens cDNA clone NHTBCae05e03 [N] | | | | | | | | | | |
| gbeh i11436 | gbeh_r11436 | 227 | — | 3 4 | 7 8 | −3.1 | — | — | — | — |
| : yf46f02 r1 Homo sapiens cDNA clone 129915 5″ | | | | | | | | | | |
| [C] [N] | | | | | | | | | | |
| gbeh t94880 | gbeh_t94880 | 228 | — | — | 3.1 | 9 | 9 1 | — | — | — |
| : ye32g07.s1 Homo sapiens cDNA clone 119484 3″ | | | | | | | | | | |
| gbeh w02997 | gbeh_w02997 | 229 | — | — | — | 3 7 | 3.4 | — | — | 5 2 |
| : za07h08.r1 Soares melanocyte 2NbHM Homo | | | | | | | | | | |
| sapiens cDNA clone 291903 5″ [N] | | | | | | | | | | |
| gbeh w37993 | gbeh_w37993 | 230 | — | — | — | — | — | −11.7 | −259 | — |
| : zc13e10.s1 Soares parathyroid tumor NbHPA Homo | | | | | | | | | | |
| sapiens cDNA clone 322218 3″ similar to | | | | | | | | | | |
| SW:SYI_CAMJE P41257 ISOLEUCYL-TRNA | | | | | | | | | | |
| SYNTHETASE [N] | | | | | | | | | | |
| gbeh w67507 | gbeh_w67507 | 231 | — | 8 1 | — | — | — | — | — | — |
| : zd40f11.r1 Soares fetal heart NbHH19W Homo | | | | | | | | | | |
| sapiens cDNA clone 343149 5″ [N] | | | | | | | | | | |
| IFN-inducible prot 9-27 | gbh_j04164 | 232 | — | — | −8 3 | 4.8 | 3 3 | — | — | — |
| : Human interferon-inducible protein 9-27 (aka leu-13 | | | | | | | | | | |
| antigen) | | | | | | | | | | |
| BAT3 | gbh_m33520 | 233 | 2 6 | 2 7 | 2.7 | 4.8 | 2.3 | 4 4 | — | 5 5 |
| : Human HLA-B-associated transcript 3 (BAT3) gene, | | | | | | | | | | |
| 5′ end. | | | | | | | | | | |
| IFN-inducible prot. 9-27 | hg1282ht1282 | 234 | — | — | −5.3 | 4.5 | 3.4 | — | — | — |
| : Human interferon-inducible protein 9-27 (aka leu-13 | | | | | | | | | | |
| antigen) | | | | | | | | | | |
| m68841 | m68841 | 235 | — | — | — | −4 4 | 7.1 | — | — | — |
| : Human L1 repetitive sequence with a region | | | | | | | | | | |
| homologous to a mouse ORF-Homo sapiens , 1423 bp. | | | | | | | | | | |
| u57009 | u57009 | 236 | — | — | — | — | — | −11 7 | −25.9 | — |
| : Human Ya5 subfamily Alu sequence-Homo sapiens, | | | | | | | | | | |
| 343 bp. | | | | | | | | | | |
| uehsf 12272 2 | uehsf_122722_{—} | 237 | — | 2.5 | — | 2 2 | 4.2 | — | — | — |
| : yg16e10.r1 Homo sapiens cDNA, 5″ end [C] | | | | | | | | | | |
| uehsf 25572 0 | uehsf_255720_{—} | 238 | — | — | — | 4 1 | 4.1 | — | — | — |
| : z134a01.s1 Homo sapiens cDNA, 3″ end [C] | | | | | | | | | | |
| uehsf 2585 0 | uehsf_2585_0 | 239 | — | — | −2.1 | 7.6 | 8.3 | 5 2 | — | 3.6 |
| : yg61f07.r1 Homo sapiens cDNA, 5″ end [C] [N] | | | | | | | | | | |
| uehsf 39737 0 | uehsf_39737_{—0} | 240 | 2.1 | 3.5 | 2 3 | 4 7 | 13 9 | — | — | 2.2 |
| : zk47f12.r1 Homo sapiens cDNA, 5″ end | | | | | | | | | | |
| uehsf 48988 1 | uehsf_48988_1 | 241 | — | 2 | — | — | 3 2 | — | −4.1 | 2.7 |
| : yh20d12.r1 Homo sapiens cDNA, 5″ end [C] [N] | | | | | | | | | | |
| uehsf 4981 1 | uehsf_4981_1 | 242 | — | — | — | 3.9 | 3 5 | — | — | — |
| : seq2173 Homo sapiens cDNA, 3″ end [N] | | | | | | | | | | |
| uehsf 49849 0 | uehsf_49849_0 | 243 | 2.3 | 6.2 | 6 7 | −8.2 | 2 9 | — | — | 4.7 |
| : yh40h10 r1 Homo sapiens cDNA, 5″ end [N] | | | | | | | | | | |
| nehsf 59701 0 | uebsf_59701_0 | 244 | — | — | — | 3 5 | 2 1 | — | −4 1 | — |
| ; zr98f03.s1 Homo sapiens cDNA, 3″ end [N] | | | | | | | | | | |
| uehsf 8069 0 | uehsf_8069_0 | 245 | 1.9 | — | 2 6 | 6 1 | 8.5 | — | — | 5 4 |
| : yh35f04.s1 Homo sapiens cDNA, 3″ end | | | | | | | | | | |
| 09.01 Known Genes | | | | | | | | | | |
| gbh 110378 | gbh_110378 | 246 | — | — | — | — | — | −17 | −20 6 | — |
| : Human (clone CTG-B43a) mRNA sequence | | | | | | | | | | |
| IF154 | gbh_m14660 | 247 | — | — | −7.8 | 22 5 | — | — | — | 4 6 |
| : Human interferon-induced 54 kDa protein | | | | | | | | | | |
| gbh m33509 | gbh_m33509 | 248 | — | — | 9 6 | 9 6 | 2 3 | — | — | −2.3 |
| : Human large proline-rich protein BAT2 (aka | | | | | | | | | | |
| HLA-B-associated transcript 2) | | | | | | | | | | |
| gbh m33518 | gbh_m33518 | 249 | — | 6 9 | — | −9 6 | 2 3 | — | — | — |
| : Human large proline-rich protein BAT2 (aka | | | | | | | | | | |
| HLA-B-associated transcript 2) | | | | | | | | | | |
| gbh u80747 | gbh_u80747 | 250 | 13.2 | — | 3.4 | 8.9 | 8.2 | 2.6 | — | 16 6 |
| : Homo sapiens CAGH3 mRNA, complete cds. | | | | | | | | | | |
| i08037 | i08037 | 251 | — | — | — | 19 3 | 12.4 | — | — | 16.6 |
| : Human RNA-binding protein TLS (translocated in | | | | | | | | | | |
| liposarcoma) | | | | | | | | | | |
| gbh s76730 | gbh_s76730 | 253 | — | 10.4 | — | −2.8 | 1 | — | — | −2 5 |
| : MM1=clone MM1 product [human, fetal brain, | | | | | | | | | | |
| mRNA Partial, 1396 nt]. [C] | | | | | | | | | | |
| TPT1 | gbh_x16064 | 254 | — | — | — | — | — | — | — | −41.7 |
| : Human translationally controlled tumor protein | | | | | | | | | | |
| (aka histamine-releasing factor) | | | | | | | | | | |
| IFI27 | hg4594ht4999 | 255 | — | — | −5.3 | 2.5 | 2 7 | — | — | — |
| : Human interferon-alpha induced 11.5 kDa protein | | | | | | | | | | |
| (aka P27) | | | | | | | | | | |
| 09.01.02 Unassociated | | | | | | | | | | |
| gbh d14661 | gbh_d14661 | 256 | — | — | — | 7 7 | 11.9 | — | — | — |
| : Human hypothetical protein KIAA0105 | | | | | | | | | | |
| KIAA0027 | gbh_d25217 | 257 | — | 6 9 | 6.6 | −6.9 | 3.7 | — | — | 21.5 |
| : Homo sapiens mRNA for KIAA0027 protein, partial | | | | | | | | | | |
| cds. | | | | | | | | | | |
| 09.02 Putative Homologies | | | | | | | | | | |
| gbeh aa504311 | gbeh_aa504311 | 258 | — | 6 | 7.6 | −3.4 | 5 2 | — | — | — |
| gbeh w45240 | gbeh_w45240 | 259 | 2 7 | 9.5 | 15.4 | — | — | — | — | 3 3 |
| uehsf 4798 0 | uehsf_4798_0 | 260 | −3 2 | — | — | 6 2 | 6 2 | — | — | 3.7 |
| : yh63c01.s1 Homo sapiens cDNA, 3″ end SIM | | | | | | | | | | |
| cyclophilin C 3.1e-248 | | | | | | | | | | |

Key to treatment groups:
4798 = collagen type I
4799 VCAM
4800 = laminin
4801 = fibronectin
4802 = fibronectin
4865 = poly lysine
5552 = VCAM
5553 = fibronectin
6892 = VCAM
5554 polylysine
6551 = fibronectin
6552 = VCAM
6554 = polylysine
6556 = fibronectin
6890 = fibronectin
6891 = VCAM

Below follows additional discussion of nucleic acid sequences whose expression is differentially regulated in the presence of an integrin modulating agent

KEANOX1

KEANOX1 is a novel 408 bp gene fragment. The nucleic acid has the following sequence:

| | |
|---|---|
| TTCACTCCCCACCCGGCTCCTTTGTGAAGTACTTCTAAGACACTCGTGCCGGTTTCCCAGTTAATCAAC | (SEQ ID NO:1). |
| ACCCACATGCTAGCCATCCCACCTCCCCAGGGCCCCGAGGTCTTGCAGATTAAAACGCACTTGCTTCTG | |
| GGTTCCAGGGGATGTCAGTGGGTGGACTGCAGGGGACTGCTGGGGGTAGTGCTCTGGGAAGCCAGAG | |
| GTCCCAACCTGGCATTATCAGCAACTGGTGGGTTAAGGGGCCATATTCTGTTTCTGAACTTTCTGGAAA | |
| ACGGGATAATAATCTCTTCTCCTCGGCAGGGTGCAGGTGGGGGATAGGTTGGGACAAGGGGGCAAAG | |
| CCGAATTCCAGCACACTGGCGGCCGTTACTAATGGATCCCANCTCGGNACCAAANCTTTGGCGTAAA | |
| KEANOX2 | |
| KEANOX2 is a novel 127 bp gene fragment. The nucleic acid has the | |
| following sequence: | |
| ACCGGTAGTGGCCCTGAAGGCCTAGCTACGCTGTTATTTATTGTATACAAGGCAAGGGGACAGGGTAA | (SEQ ID NO:2). |
| GGAGTGCGAGTCATGTCCAATGATAGGTAAGGGCACGCGAGTCATGCATCCACTGTACA | |
| KEANOX3 | |
| KEANOX3 is a novel 1655 bp gene fragment. The nucleic acid has the | |
| following sequence: | |
| GTCGACTTTTTTTTTTTTTTTTTTTTTTTTTTTTGAGTHAGAACTCAAACTTTATUGTGCTGTAAAGGG | (SEQ ID NO:3). |
| GAAGAAAAGTGATTAGTAATTATGTACATGACGTAACAGAATTAGTTCTTACCATACAGAGCAACATA | |
| CCAGTACTTTCAACCATCTACTTTTACAGTTTCAATTTCACAACAGCATACCCGAGACTACTTCCCCAT | |
| ACATGCGTGACTAATACACTCTAAATCCCAACGACAGTAATTGTTTGCATCATCCCCAAGTCATTGGTT | |
| AAGAACTGAACTACCATGTCAACATATTTTTAAACATTTAAAAAATGGAACCCAGAAGACAGAAAGA | |
| TCCCAACGATAAAAATAACCATGATTTTTAAAATTTCAAGTGAAACTTAAAGGGAAGAAGGAAATGAT | |
| AAAAAGCCATTGGTATTACTTTACCAATTCAATAGACGCAGCTCAGAACCAAGCAGGGTTTTCAGATC | |
| ACACATGTAGTAAAGGCACCTCGCCCACTCTCTCCCCACAATCCCCATCCCCTTCCCTTTTTAGGTCTT | |
| GTTTTCCAAACTGATTTTAGGTGATAGTTTCCCCTCTAGTCTTGGCTCATTTAAAAAGCACAAACATTC | |
| AAATTAAAAATACAATAAATTATTCATTCTAACCTTAGCAAAGGTACATGGATTCTGTCTACCAAATAT | |
| TAATTTTACAGTGACCACAAAAGGTTATTTGGTTGTTACAAAGACTACAATTTGAAGATACTAGATAG | |
| GCAAAAATTTTGCCATTATTAATATCGACCAAACACAGAAAAGACAACAGATGTGTTTCTAAGGCACG | |
| ATTTACATACTAAATTCCCTTGAAATTCAATGCAATTTCACTTCGCTCATGATTTTCAATCACAAGACA | |
| CCTGTGACAAATGAGGAACAAATCCAGAAGAGCCCATCTAATCTCGTCTTAAGTGGTCCCTTAAATAC | |
| GTTAAAAGCCCACAATATAGGCATTTACAAGGCTTTTCTAAGACTTCCAGCTCCTGAAAAAATCACAC | |
| CTTCACAGTTTCAAATCAACCACCTAAGTTGCTAAGGGGCAGCGAAGGATGCTGATCTGCTGCGTATG | |
| CAAGTCTGACGCCCATCTTTCAAGTGACTAATTCACTTGATAACACCCACACCCCAAACAAAACAACC | |
| CTCTGAAACAGGCTGTCTTGGCCACCAGGAGGAAGCTGGTAAAGACATTTCCCCTGCCTGTGAGATGG | |
| CATCACACACCCCTCCCTTTGGTTCTCGGAAAACTGGTGACTCTGTGTCACCTGTTTTCAGTTAACACTT | |
| CTTCAGTCTTCCCCACCTTGTAGCAAAGCAAAACAATTTTTGTTTGTTAGTTTGTCTGTTTGCACTTAAA | |
| GTTCTAAGCACTTTGGAAAGTTTCTAAGCAACTTCTCACTTCCAAGCAACAACTTAACCAACACTAACA | |
| ACTTACTATTATTAATTAGTATTTTCTTGGCTCACCCACGCACTAAATTCCCCAAAGCCCCAGAGAATT | |
| CCCTTCAACCTGCATTTCCTACCACCCCCACCACCCACACTGTATCTCTAACCAACCCTCTCCCCTTCTT | |
| CACCCTCATCCCTCCCAGTACCAGTGCCAAGCTGCATACCCCCATCCCCCACACTGCAACGCCCACGG | |
| GACCGGN | |
| KEANOX4 | |
| KEANOX4 is a novel 57 bp gene fragment. The nucleic acid has the | |
| following sequence: | |
| GGTACCTTACCCCAAAGTAGTGCGGTTGGTTCTGCCTTTACACAGGATACAANATCT | (SEQ ID NO:4). |
| KEANOX5 | |
| KEANOX5 is a novel 896 bp gene fragment. The nucleic acid has the | |
| following sequence: | |
| ATTATTCCCGCGAATATGTTTTCCAAACTTTTAGATTTCTTGCTGAGGAACACAAATTATTCTT | (SEQ ID NO:5). |
| AGGTTTGGTCATTTATCATAATCCTAAACTTCGTGGAGGCTTTGTTCATTTTTAAAAAGTTCTT | |
| TTTTCTTGTTGGATTGGGTTAATTCCAGAGCCTTGTCTTCGAGTTCTGTAGTTCATTCTTCTACT | |
| TGTTCTAGTCTATTGTTGAAACTTGCCAGTGTAUTTGTATTTCTCTAAGTATGTCTTTCACTTC | |
| CAGAAGTTGTGATTGATTTTTATTTATGATATCTATTTCTCTGGAGACTTTTTCATCCATATCCT | |
| GTATTATTATTTTAATTTCTTTAAGTCGGTTTTCACCTTCCTCTGGTATCTCCTTGAGTAGCTTA | |
| AATAATCAACCTTTTGAATTCTTTTTCTGGCAATTCAGAGATTTATTCTTGGTTTGGATCCATTG | |
| CTGGTGAGCTAGTGTGATCTCTTGGGGATGCTATTAAAGAATCTTGTTTTGTCGTATTACCAGA | |
| GTTACTTTTCTGATTTCTTCTCATTTGGGTAGATTGTTTCAGTGGGAAAGATCTGGGACTCAAT | |
| GGCTGCTGTTCAGGTTTTTTGTCCCATGGGGTGATCCCTTGATGTGGTTCTCTCCCCCTTCCTCT | |
| AGGAATGGGGCTTCCTGAGAGCCAGACTGCAGTGATTGTTACTGCCCTTCTGGGTCTAGCCAC | |
| CCTTCAGGGCTACCAGACTCCAGGGTGGGCAATGGGGAATGTCTGGCAAAGAGTACTGTGAT | |
| GTGATCCACCTTCAGGGCTCCCAGCCATGGATACCAGCACCTGCTCCATTGGAGGGThGCAGG | |
| GGAGTAAAGTGGACTCTNTGGGAATCCNTGGTNGTAAThTAGGGCACTGGGTTTTCTTGAAT | |
| KEANOX6 | |
| KEANOX6 is a novel 2958 bp gene fragment. The nucleic acid has the | |
| following sequence: | |
| ATTGGCACGAGGACAAACCAGGAGAGGGGGTTCTGGAAGCCAGAGAGGGCGTGCACACACA | (SEQ ID NO:6). |
| GGCGGGGGAGGGAGCCTGGGTGCAGCAGGGTGGGCCACTGCGACTCACCGAGGGCCAGCGG | |
| CGCTGGGGCCAGGGGCTGGAGTTTGGAGAGTGAGCAGAGAGCACGCAGGGGCTGTGAACACC | |
| AACTTCTCTTTCAGGTCAATTTGCTGCAAAGGGGAAGGTAAAGGTGGAGGTAACTAAGGAGC | |
| AGGGCAAGAGAGGGGTGTGGTTTTCTGTAGGGTCCAGCCCTACGGGGGCTTAGCAGGTGTCCT | |
| CCCCGTGTGCAGAGATCAGAGATTGTAATAAATTTTGATACTTTTTTTTTTTTTTAAAGAGAAA | |
| ACAGCTGGGCCCGGGGGACCACTACCATCAAGACGCGGAGACCGGTAGTGGCCCTGAACGGC | |
| TGGGCTGCGCTGATATTTATTGCATACAAGACAAGGGGGGGACAGGCAGGGTAAGGAGGGTG | |
| AATCTTCTAAGTGATTGACAAGGTGAAGCAAGTCACGTGATCACAGGACAGGGGGCCCTTCC | |
| CTTTTAGGTAGCCGAAGCAGAGAGAGAAGGCAGCATACGTCAGCGTTTTCTTCTATGCACTTA | |
| TAAGAAAGATCAAAGACTTTAAGACTTTCACTATTTCTTCTACCGCTATCTACTACGAACTTCA | |
| AAGAGGAACCAGGAGTACGGGAGGAGCATGAAAGTGGACAAGGAGTGTGACCATTGAAGCA | |
| CAGCACCACAGGGAGGGGTTTAGGCCTCCGGATGACTGCGGGCAGGCCTGGATAATATCCAG | |
| CCTCCCACAAGAAGCTGGTGGAGCAGAGTGTTCCCTGACTCCTCCAAGGAAAGGAGACTCCCT | |
| TTCGCGGTCTGCTAAGTAACGGGTGCCTTCCCAGACACTGGCGTTACCGCTTGACCAAGGAGC | |
| CCTCAAGCGGCCCTTATGCGGGCGTGACAGAGGGCTCACCTCTTGCCTTCTAGGTCACTTCTC | |
| ACAATGTCCCTTCAGCACCTGACCCTATACCCGCCGGTTATTCCTAGGTTATATTAGTAATGCA | |
| ACAAAGAGTAATATTAAAAGCTAATGATTAATAATGTTTATTATAATGATTGATAATTGTTCA | |
| TGATCATCTCTATATATAATTATGACTATTATTATTCTATTTTCTTTATTATACTGAAACAGTTT | |
| GTGCCTTCAGTCTCTTGCCTTGTCACCTGCATAATCCTCAGCCCACAACTGGGACTACAAGTGC | |
| ATGCCACCAAACCTGGGTAATTTTTTCTTTCTTTTTTTTGTACAGATGTGGCCTTACTATGTTGC | |
| TCCGGTTAGTCTCGAACTCCTGGCCTCAAGCTATCCTCCTGCCTCAGCCTCCCAAAGTGCTGAG | |
| ATTACAGGTGTGAGCCAATGCAGCCACCCCAAAGGTGGTGTTCAAAGTTGATTTCTCATCCAA | |
| CTCTGATTCTAGACTGAATAGCACAGACCCTCAAATGTGAGGTGCTAACTAGCAATCCTGGAA | |
| TCCAGAGATATACCCATGATGGACAGCCCAGAGAAAGCCCCAGTGCTGGGGCAGAATCCACC | |
| TGCTCAGGTGCAAGAGGCCTAGCCACTTGGGAGTGTTCATCACATGCACATTATCTGAGGTTA | |
| GGTGTGGGAATGCCTCAGCCAGGTGTGCAGAGATGTGTATGGGGAGCAGGTGAGAGACATGG | |
| GCACCTGGACTCCCCAGCAAAAGCTTCAAGACAAGCTTAGTGCAACCAATCCCCTGGGCTGTT | |
| CATAACAACCCCTCTCTGGTCCTGCAGGAGGGAGGTAGGCTCATATAGAGGCTTCCTGGCAGG | |
| GCCCTACAGCTGGAGACAGAGGCCACCCACCTAAGGACCTGCATCCTGCAAGGAAGCCAACT | |
| CATGCCCTGAAACCCAAGTCCTTACCCAAATGGATCACATCCCCAGATGGATCACAAAGCCAT | |
| GGGGCTTTCCCATGTGCTGTCCCTGCAGGCCTCTTTCTGAGGGGCATCAGGTACCCCCCCAGTC | |
| CTCCATCGTGGGATCTAGGGCCTTGTGTCCAAATGCCACCCTTTCCTTGGAATGAGCCCATATC | |
| CGTGGGAAAAAGAAAGTCTGGGGGCATGGCAGGAGTCCGGGGGAAAACAGCAAAAAGCAGG | |
| AGGACAGGGCAGAGAAAGCAGGGAGAGGGGAGGAGACAAGAAGAGAGAGGGAGAATGAGC | |
| ACCCAGGCCTGGCCAGGGGATGCCTGCAACCATGCGGATACCCCACACCAGGGCTGGGATGC | |
| CTGGTGGGAAGAGCCCAGGTGTGCTTCCTCACTAGCTCACCTACCACAGGTCTGCTGGGGCAG | |
| AGCGTCATTTCAGAGGACAGCGTGGCAAGAAGCATCCAGGGCCCGGGAAGAACAGAGCTGG | |
| AAGGTGTGCGGCTGGGCAGTCCTCCCACTCAGGCCCACATTCTGCACTGCTGAGTCTCTTTGG | |
| GGGCTCAGCTGGCTCAATCTTTGCAGTCTACAGGATTGAAGGTCAGTGGAAATGTGAACAGA | |
| AAATAAGCAGGAAGTCCATCTATCCCTGCTAACCCTACCTCTGGACACACGGAATGCTCAGTG | |
| CAGTGGGAATAGGCTCCAGGAAGACCATGGAGGGAGATGCTGCACAGTGAACAGTGAACCTG | |
| GCTTTCTCATGGCCTTCCACTAGGTCACTGGCACACTGTGCTCCAAGGAGCCCCAGGCTGTAA | |
| TAAGTCACACTTGAGAGTTCATGGAAGCGTCTGAGAACCATGTATTTGAGATGGAAGTTCACT | |
| TGAAATAATAAATTAATGCAGATAAAAAAATATTGGGGGAATTAAAGGGCCAGGCACCATGG | |
| CTCACGCCTGTAATCCCAGCACTTTGGGACGCCAAGGCGGGTGGATCATGGGGTCAGGAGAC | |
| CGAGACCATTCTGGCTAACACGGTGAAACCCCGTCTCTACTAAAAATAGAAAAAAANTAGCC | |
| GGGNCGCNACGAGG | |
| KEANOX7 | |
| KEANOX7 is a novel 319 bp gene fragment. The nucleic acid has the | |
| following sequence: | |
| TCCGGACTGGAAGAACTCATCATTCTTCANACTGGAATTTTATCGGCTCTTACAGGTTGAAAT | (SEQ ID NO:7). |
| CTCCTTTCATCTTAAAGGCATTGACCTACAGACAATTCATTCCCGNGAGTTACCAGACTGTTAT | |
| GTCTTTCAGAATACGATTATCTTTGACAATAAANCTCACAGNGGCAAAATCAAAATCTATTTT | |
| GACAGNGATGCCAAAATTGAAGAATGTAAAAACTTGAACATNTTTGGATCTACTCAAAAAAA | |
| TGCTCAGTNTGTCCTGGGGTTTGATGCATTTGGCATTGGATTTGCTGGCNTCTNTTATTCTGGT | |
| CTN | |
| KEANOX8 | |
| KEANOX8 is a novel 276 bp gene fragment. The nucleic acid has the | |
| following sequence: | |
| CAATTGCTTTGGTCACAAAGGCAGGTCTAACAGTTTAATTCCACGCCAGAGAGGAGGGGACTTTTCAT | (SEQ ID NO:8). |
| GTGGCTGCTGATTTGGAATTTGGTTCCAGGACATAAATCTGAGGACACATGGGGTAAAATAGCCCGCT | |
| TTCTAAAGAAACCTGGAAACAACTGGGGTAAGACATCTTCTCAGGGTTCCTTGCCCCAGCTCTCTACA | |
| CACCCCACCTCACTCTACACGCTGAACCCTGGGCACCATGGATTCAAAGACATCTTTGCCACTGTGAG | |
| ATCT | |
| KEANOX9 | |
| KEANOX9 is a novel 125 bp gene fragment. The nucleic acid has the | |
| following sequence: | |
| RGATCCAGCAATTCTTTTCAGAATCTGACAGTTACCCCTGCAAATGTAGAAATGACATGTATTCACTGA | (SEQ ID NO:9). |
| AGAGTTGTTTCTAATATCTGAAGACTGGAAACAACGCCAAGTGTCCACCTACGCGT | |
| KEANOX10 | |
| KEANOX10 is a novel 1328 bp gene fragment. The nucleic acid has the | |
| following sequence: | |
| GAAGCGGCGGGCGGGGTGGAGCAGCCAGCTGGGTCCGGGGAGCGCCGCCGCCGCCTCGATGGGGTGT | (SEQ ID NO:10). |
| TGAAAAGTCTCCTCTAGAGCTTTGGAAGGCTGAATGCACTAAACATGAAGAGCTTGAAAGCGAAGTTC | |
| AGGAAGAGTGACACCAATGAGTGGAACAAGAATGATGACCGGCTACTGCAGGCCGTGGAGAATGGAG | |
| ATGCGGAGAAGGTGGCCTCACTGCTCGGCAAGAAGGGGGCCAGTGCCACCAAACACGACAGTGAGGG | |
| CAAGACCGCTTTCCATCTTGCTGCTGCAAAAGGACACGTGGAATGCCTCAGGGTCATGATTACACATG | |
| GTGTGGATGTGACAGCCCAAGATACTACCGGACACAGCGCCTTACATCTCGCAGCCAAGAACAGCCAC | |
| CATGAATGCATAGGGAAGGTGCTTCAGTGTAAATGCCCAGCCGAAAGGGTCGACAGCTCTGGGAAAA | |
| CAGCTTTACATTATGCAGCGGCTCAGGGCTGCCTTCAAGCTGTGCAGATTCTCTGCGAACACAAGAGC | |
| CCCATAAACCTCAAAGATTTGGATGGGAATATACCGCTGCTTCTTGCTGTACAAAATGGTCACAGTGA | |
| GATCTGTCACTTTCTCCTGGATCATGGAGCAGATGTCAATTCCAGGAACAAAAGTGGAAGAACTGCTC | |
| TCATGCTGGCCTGTGAGATTGGCAGCTCTAACGCTGTGGAAGCCTTAATTAAAAAGGGTGGAGACCTA | |
| AACCTTGTAGATTCTCTTGGATACAATGCCTTACATTATTCCAAACTCTCAGAAAATGCAGGAATTCAA | |
| AGCCTTCTATTATCAAAAATCTCTCAGGATGCTGATTTAAAGACCCCAACAAAACCAAAGCAGCATGA | |
| CCAAGTCTCTAAAATAAGCTCAGAAAGAAGTGGAACTCCAAAAAAACGCAAAGCTCCACCACCTCCT | |
| ATCAGTCCTACCCAGUGAGTGATGTCTCTTCCCCAAGATCAATAACTTCGACTCCACTATCGGGAAAG | |
| GAATCGGTATTTTTTGCTGAACCACCCTTCAAGGCTGAGATCAGTTCTATACGAGAAAACAAAGACAG | |
| ACTAAGTGACAGTACTACAGGTGCTGATAGCTTATTGGATATAAGTTCTGAAGCTGACCAACAAGATC | |
| TTCTCTCTCTATTGCAAGCAAAAGTTGCTTCCCTTACCTTACACAATAAGGAGTTACAAGATAAATTAC | |
| AGGCCGCTCCCAGCTAGTTTGTATCCATAATAATTTAAAAHTTTAAAAAATCATTGAGTGAATAAATT | |
| AAATTTACAAACCAATAATTGATTTTCTTCATTTAAA | |
| KEANOX11 | |
| KEANOX11 is a novel 1971 bp gene fragment. The nucleic acid has the | |
| following sequence: | |
| CCATAGTATCAGTTCAAGCTAAGAAAGCCTTAAGAATTCAATAACTCAAAATCTCTHGAGAT | (SEQ ID NO:11). |
| TTAAAGTTAAAAAGTAATATGTTTCTAATAATTTCTAATGTGCAGGATTAATGACAACTGTATT | |
| TTGCATTGGCAAATTATCTCAGCCAGAAGAGATTCAAATGATTTTCGTCAATGCCAGTTTATA | |
| AACTAGCAGCACATGTCAATGCCAAGCTTGTGAATGGATTGGCTGAGAAAAATTATATGAGTT | |
| AGAAGGGATCTAAGTGCTATACTGGTCACTGATTCTATTTCACAAAAGCTACCATTTTTAAAA | |
| GCTGAAAATGTAATTGGTGTTGACAAAAACATGTTAAGTTCAATTACTCTGTATAGCTAAATA | |
| TAGAAAATTGGTTAATGTTTAAAGCAACTTCTTATTTGTTAAGAAGATAATTAAAAATAGTTA | |
| AAACTGAACAATTAGCTATACTTTTATATTATTAAAATCCAATGAATTTAACAAATGAAACCC | |
| ACAGGAAAAGAGATGATATAATTCAAGGCATAGATTTGACATGTCAGAATACGGCAAAAACA | |
| ATAAAGATAGCATCAGTAAAACAGTTGTAAAATAAGTAAACAATTTTCAAGTGTGCAAAACT | |
| AGTCAGGAATAAAAGTGCTATGTGTGGATACCTGGTTTTCAAATATTTGTTTCAAAAACTTTC | |
| AGCTGAATGGGTGGAAAAACTAGAAAGTATTATATAAGTAAAAATTTTAAAGAATTATATAA | |
| AACCCTACTTTATTATCCTACAATTATGGCCTTGTTATGTCACTTGCTGAATCTGAGGCCTANTT | |
| TAATTACCAGATACAACACAAAAGGAGGTAAACACAGTACATCACAAAGGCTGATTTATCCT | |
| GAACCGTAAAAAGGCTCCTTTCAAGTATACTGTGGATTTGTGTTTCATCTTATATCTCAGGCAC | |
| TCTAGGCTGTTGATGAATTTGAAAACGGACAACATGATTTTCTATAATTTCTAATATACATATT | |
| TAGTACATCTTGCAGAACTGCCTTTAGTAGTCAATATTTGGAAGCAATTGTTACAGAAAATAA | |
| AAACAATTTAAATTCATGAAAGTAAACCCCATCACTGCCACAATTTAAATAATCATTTTAATG | |
| TGTAAATCCTGAATATATCATGTTAGAAGATGAAGTCGATGTATTTAGCATAATCTGTCTTGG | |
| GATATTTGACGTGTAGATGACTAAAAATACACTTGTGGTCGTTTCTTCACTTTGTATTTTAGTG | |
| ACCCGTACCGCACCTCTTTGCGGCCTTTGGTGGTTTTCACAGATTTCATGCTTTGCGATCTTCG | |
| AAGCCCTTTGTGTGITAACATCTCATTATCTGAATATGGCCCCTCTGCTTCTTCATCTGTTTTCT | |
| CATCTCCGTCTTTCAGTGGAATTCCATAACCTGCTGATTCACTGGGGGCTGGTTTACTTTTCTIT | |
| TTTGCTTCTGGTAGAGGTTGGTATGGTCTTTGTCCCACAGGCTCATCACCTTGGATAGTTGTTA | |
| AATCATGCATACTGAAGCTTCTTAAACGTTCAGTTATTGCTCCTTGGCTCTTTACTGCTGCAGT | |
| AACAGCAGCAGTAGCTGCAAGGTTTAAACCTTGCCGTCCAAAGTTTACCATGGTTTCATAGCC | |
| TCGTTCCTTTGCTTGTACAATATAATCATCAATCTCCCTTTCCTTTGAAGAAAGAAGTGGATGA | |
| AGGAATTTTCTATATATTAAACTTGCTCCTTTGGTATAGGGAGAAAGCAGCCATATGACAAAA | |
| GCAATCTTCAGCTCATAGTACAGGGGAAACCAAGCAACTGTTTGATCGGCTACTGTTTCAATC | |
| ACAGTATAGAGAGCAAAAACAATCCAGTACATCATCCATCGAACATATTCCTTCACGTTTTTT | |
| GTTTTCACAGCTTTGTATGAATAATATGCAGGATTAAAGCATTCCAAACACCAGCACCACGGC | |
| TCTTGAGAT | |
| KEANOX12 | |
| KEANOX12 is a novel 4027 bp gene fragment. The nucleic acid has the | |
| following sequence: | |
| TTTTTTTTTTTTTTTTTTTAATTAAAGGAGTGAATGTTTTATTCTTTAGTGTTTAATTGAATACATAACAA | (SEQ ID NO:12). |
| GTCACATAATCAATGATTCATTACTTCACACACAGGAAGGAAAACAGTAGCTAGTATTTCTGTTATAC | |
| AACTGGTTGTGAACAGGAAGAACAAAAATCTTTATAGCATTAGATGGTTGAAGAAATTCTCAAAGTTT | |
| GGGCATGTCTGAAAAGACTTTGACTGCATCTTTTTTCAATATAAAGGGATTTTTTTCTTACATTTCTGTG | |
| TCATTAGAAAAATCTGATAATCTTATAAGATTTTTTCTGCTCTTCTTACATTTAGAGAAAATGGTTTAAT | |
| GATATGTACTATTTAAAAAATCAGAATCCCAGATAACTCTGTGTGAATCTTACATTGTCTAGCAGATAA | |
| GGTTTTCAGATGCCTGGAAAAATTTAAACAACATATAATTTACATCTTCAAAATTAACATCCAGATGG | |
| ATGTTTTAATCCTTTGTCACTAAATGGTTACTAACGCTGTTTGAGTGGTCATAAATGAATTGTCATTTGT | |
| ATTCAATAGTAATAGCAAAATTTACCTGCCTATAAAGTCGCAAAAACAGTTGTATAATAGTTTACATT | |
| ACAATTAATGTACCCATACCTCAAAACACATTTAGAATTTAAAAAAACTGGCTTGGAAAAAAATCACA | |
| AAAAATTTAAGTGACAACAAATCTTGATTAACTAGTCCATCTTCCTACCACACATGATTATACTCTAAT | |
| GTAGATATTCTGGTTGAATTTCTTCCAACCAAGTTTGAAGGGCCCCAGTAGAAAATCAAGTGTTAATA | |
| CTTTCAGATTTTTATTGTCCAAAACTGTTGACAAAGAAAAGCTAACTTCAACATAACTTGTTTCTGGCT | |
| ATACAAAGACCTATTTCAGGTGCTACAGATACTGAAAATAGGAGTATTTTACTTATTTCTCAAGAGAC | |
| AGACTTATGCCATCTAAGAAAATGAATTCAGTTTTTAAATGATTTATAGTAAGGAAAAACAGGAAAAC | |
| AATTCCAAACGAAGGATTGACTCTGATAGGCTTACAACAAAAATATTCAGTTTACTATHTAAAAACT | |
| AAATTGAGTAAAGTATTTGTGATTTAGACTATAAAATAAATTCTCAAGGAATTCAACCCCTCCAGTCA | |
| ATACTTTTTCTTAATGTCTGCATTGTAATTTAGCATTTTGCATGTGGGAGTACACAAATGAATTGAATA | |
| TTGGATCAGAATTTACCCCTAACTTGAAGAGTAAAAAGTTATCAAAGTTTCACCTTACATGGCTTTATT | |
| GAAATAACATTCCATCGAAAATTCCAATAAAAATTGGAATATATTATGAGCAGTGCCAATTGCTCATT | |
| TTGTCTGATATTAACAGATTATGCATTTCCTCAGAGAAGCAGTAGGTCCCATATATACAGACATATATA | |
| TGGCTCTGGTTTGAAAGAGAAGACAATTTTGTCTAGTATTATGATTTATCACTGTGCTATCAATCAAAA | |
| AAACCAAGTAAGAAATACAATTAAAACAACATTGAAAGGGAAAAATTTTTAAATCTCACATCTTCAAG | |
| GAATATTTTCTACAGCATATCATTTGAAAAAGTAGAATGAATACTGTTCATGGTCCACATGTATTAAAC | |
| AAAAAACATGTCAATTACTTGGTGCAAACACACAGAACCTGGTACCTGTTTTTTTTTTTTTTTTAACCA | |
| CCATTGTCTACACCTUTTAAAAATTAAGTTTGTTACTAAAAGTCCAATGTCATTCACTTGTATTTATGA | |
| TCATCAAATGGTAATTAGGGCAACATATGTAAATGCATGCCTCTGAATCAGATTCATGCAGTGTTAATT | |
| ATCTGAATAATTTATGACATTCTCCCAGGTTATTTGAATGGTATCTTTGGAGGGCTTACTCAAATGAAC | |
| CCACAATACCTCCACTATTACAGCTTATAGGAAATTACAATCCACTTTACAGGCCTCAAAGGTTCATTC | |
| TGTGGCCCAAAGCCCATGGAGGGGAAGGGATCTAAAGGTGCTCATGTCAAGTTATTTTACTTGTTTTTT | |
| ACTGTCTACCCAATGTAAAATGAATTTTCTTCACAGCCTTTACTTTAGGCTAAAAATAGACAGTTTTCT | |
| TGTATTGACTTTAACTTTAGAGGGGACAGTCGTCCCTGGAAAAAGAAGTAGATTTACTGTCCCTAACC | |
| ATTAAATTCCCAGTCTTTCAAATAATGTGCGGGGCGGGGGGTGCTGGGTGTGACAGGGTGGAGTGAAT | |
| AAGATGCCTATGTAGAAGACAGTGGAATAGTTCACCTCAAAGGTTTACAAGATAACATTTGTAAGACT | |
| TGAGTGACAGAGGCAAAATTTTAGCAGTTTCTCAAGACTCTTGGAGTTGAGCAGATTTGAGAAGGGAA | |
| ATCAGAATCGCCGAGCCTTGCCATCTGTCAGTGGTTTTGGTCTGTCCCAAGATGTTCTCAATCATGTGC | |
| TTCCTGTTGAACAATCTAACAAAATACCATACGGGGTAGAGAAATCTCTGCAACACTGACTTTTGGTC | |
| AGTGAATCAGAGTCCTAAATTATTTCCAGGCTTCAAACAGATGAATTCTGATGCATTACTTAGTCTGAG | |
| ACCAAACTCAACATACAAGCAAGCACAACTTCCTAGATTTCTGCTTCCATTTCGCCTAAAATTACAGGC | |
| TTTGTATATGAAGTCAATTTTACTTCTCAAAAAATGTTAGTCTAATAAAAGACCTGAAATCTATTTCCC | |
| AACTAAAAAAAAATTAGTGGATTAAAAATACAGGTACCTTGACTTTAAGTAGACAGTTAACAACATA | |
| TGATTAGTAATACAACTAATACTCAAGAAAAAAAGTATAATACAGCAAACTCAGTTTTACAATTATGT | |
| ATTTTTTCCTAACCCAATTCCTCGATAAGGATGTAAGAAACTCATCAAAACATGTTGCTTTAGTATATA | |
| AACCAGCTGATGCAAATTACTGGGGTAATATAGGAAATTTTATAGCATTAGTAAATGAATTAAAAAAC | |
| TAAATGATTCATGTAAAATGTTTATATATGGTATATACAGCTTGGATCACATGAGTCTAAGAATAAAA | |
| CTGTCGATTTGTAACATCTGTATGGACATAGTAAGATACAGTACGAGATTCATTTTTAAAAACAAGGA | |
| AAAATAGCACTAATTTAAACACTGAAAACAGCTCTTACAAAATCCTTAAAAACGTACATCAGAGAAA | |
| GATGCTGAAAACAAAACAAAATAGGCTTCTCTGTCATCACAGAATATCCAAAGGACACCTCACAGCAT | |
| GGAGCATGACGAGGTTTATTTCCCTTCACTTATGGTTTACATAGGATATGCATTGGGAATTTATTCCAT | |
| GTAAGAGTAAAGGCATAAGGCAACCAGGTCTCCTTCTGTTTCTGTTGCATTTACCTATTATTGGACCAT | |
| TTTATTGCTCAGCTCACTTTTAAGTACTTATTTGCCTTCATTTAAAAATATTCCATTTTACAGCTATCTA | |
| CAGATAGGCAGCCCTTGGTGCTGAGTTCTCTGCAGCTCGTCATTTGAAAGGATTCCCACTTAACTTCAA | |
| ACAAACTGCATGAAAGTGGCATTTTCAGGTTTTCAGGTTTGCCCAGAAGTCAACGTTTGCTGGGCTTGG | |
| AGAAAATAAGGATTTTTAATAAGTGATCTTAGGTCTAAGTAACCAAAATGAAACTTCTTTTGCGCAAT | |
| AGATTCCAAAATCAGGATTTATAATCAACAAGAAAATAAAAGTTCAAAAACTAAAACCCTACTGGGT | |
| AACTCTTTGGTTAGATTATATATAAGCATTAACAACTCTAAGTTTAGGACTCAAAACAGTAAGTTAAGT | |
| AAACTGCCAGGTAAGAGTAGGTCTGTTATTAATCTTTATAAAATACTTGGCATGTTTGCATTTACTATA | |
| TGATATTTTTTAAATGTGCATATACTCAAACAGATGGTACAAATTTCCAACTTTCAAATCATAAATGAC | |
| TATGTCTGCTTCTCCCTTCCCACCCTTCCTACCCTTTCCCTCGTGC | |
| KEANOX13 | |
| KEANOX13 is a novel 157 bp gene fragment. The nucleic acid has the | |
| following sequence: | |
| GTCGACGCAGGGGGGCAGTGTTCTANAACATAACACACCTGCTTCCGAGGACTAGCACACTC | (SEQ ID NO:13). |
| CCANCAACACGGGGATTTTAGGCCATGCCACACTGTTTGCTACCAGCTTTTTCCTACTTAATTT | |
| ACATTTCCATGTCATTATTATTCAAAAGCTT | |
| KEANOX14 | |
| KEANOX14 is a novel 1432 bp gene fragment. The nucleic acid has the | |
| following sequence: | |
| TTCCCGCACCTGGTCCCGGCCAGCCTGGGCGTGGCCGCCGTGCAGGCGCAATTCTCCAAGTGA | (SEQ ID NO:14). |
| GGGCGGGCCTGGGCCTGGGGCGCGACCTCGGCCCGGCCTCCCTTCGCTCAGCTTCTCCGCGCC | |
| CCTGCTCCCTGCGTCTGGGAGAGCGAGGCCGAGCAAGGAAAGCATTTCGAACCTTCCAGTCCA | |
| GAGGAAGGGACTGTCGGGCACCCCCTTCCCCGCCCCCACCCCTGGGACGTTAAAGTGACCAG | |
| AGCGGATGTTCGATGGCGCCTCGGGGCAGTTTGGGGTTCTGGGTCGGTTCCAGCGGCTTTAGG | |
| CAGAAAGTGCTCGCTCTCACCCAGCACATCTCTCTCCTTGTCCCTGGAGTTGCGCGCTTCGCAG | |
| GGCCGATGTAGAACTTAGGGCGCCTTGCCGTGGTTGGCGCGCCCCGGGTGCAGCGAGAGGCC | |
| ATCCCCGAGCGCTACCTCCCCGGAGCGGAGCACGCCGGCTCCCAGTACTAGGGGCTGCGCTCG | |
| AGCAGTGGCGGGGGCGGAGGGGTGGTTCTTTTCCTTCTCCTCCGCCAGAGGCCACGGGCGCCC | |
| TTGTTCCCGCCGGCCAGGTCCTATCAAAGGAGGCTGCCGGAACTCAAGAGGCAGAAAAAGAC | |
| CAGTTAGGCGGTGCAGACGGTCTGGGACGTGGCAGACGGACGGACCCTCGGCGGACAGGTGG | |
| TCGGCGTCGGGGTGCGGTGGGTAGGGGCGAGGACAACGCAGGGTGCGCTGGGTTGGGACGTG | |
| GGTCCACTTTTGTAGACCAGCTGTTTGGAGAGCTGTATTTAAGACTCGCGTATCCAGTGTTTTG | |
| TCGCAGAGAGTTTTCGCTCTTAAATCCTGGGGGTTTCTTAGAAAGCAACTTAGAACTCGAGAT | |
| TCACCTTTCGTTTCCCTTTCCCCAAAAGTAGCGTAACCAACATTTAAGCTTGCTTAAAAACGAA | |
| AACCAACCGCCTTGCATCCAGTGTTCCCGATYITACTAAAATAGGTAACCAGGCGTCTCACAGT | |
| CGCCGTCCTGTCAAGAGCGCTAATGAACGTTCTCATTAACACGCAGGAGTACCGGGAGCCCTG | |
| AACCGCCCGCTGCTCGGCGGATCCCAGCTGCGGTGGCGACGGCGGGAAGGCGCTTTCCGCTGT | |
| TCCTCAGCGGGCCGGGCCCTTGACCAGCGCGGCCCGCAGGTCTTCCTTCTCGCCGTCTTGCAG | |
| TTGAAGAGCTACATACGTAGTCAGTTTCGATTTGTTACAGACGTTAACAAATTCCTTTACCCAA | |
| GGTTATGCTATGACCTTTCCGCAGTTTACTTTGATTTTCTATGTTTAAGGTTTTGGTTGTTGGTA | |
| GTAGCCGAATTTAACTGGCACTTTATTTTACTTCTAACCTTGTTTCCTGACGGTGTACAGAATC | |
| AACAAAATAAAACATTTAAAGTCTGAAAAAAAAAAAAAAAAAAA | |
| KEANOX15 | |
| KEANOX15 is a novel 143 bp gene fragment. The nucleic acid has the | |
| following sequence: | |
| ACTAGTCAACTTTCCAATTGCCTATGGCTATGCCCCTCTTTTCTTAGGAAGCATAGACAGGGAAGCCTA | (SEQ ID NO:15). |
| GGAGCTCACCTGTTTTTACCGGCAGCAGGAAAAGGATGTTTAATGGTGCCATGACAAAACTGCTGCCA | |
| TGATCA | |
| KEANOX16 | |
| KEANOX16 is a novel 3679 bp gene fragment. The nucleic acid has the | |
| following sequence: | |
| TTTTTTTTTCATGCGGAAACCCTTGTGATTTGATACATTACTGGCAGTGTGAAGCTAAGGCTATGTGAC | (SEQ ID NO:16). |
| TAATATTTCAGCAAAGGATAAATTTTTTTTTTTTTTTTTTATAGTAGCAATAGTTTCTTTTTTTATTTTAA | |
| TATACTTTAGGAAACAATATACAAAGCTGCGCTTTCCTGCTATTTCCAGATAACAGCAGGAAGCTCCA | |
| ATTACACAAATTTAGCGTTTTGTGATGGCTAAGAACAGCAGTCAGCACCAGACTTGAACAGTTAGCTA | |
| CAACACAAACATCCTTCCAAATGAAATGTCATAATAGCAAGACAGGTAAACCAGGGTTTAACAAATG | |
| ACAACCACTTGGGAAGTGACTTAATCATTCTTGTTGAATTTCTTATTTCATTAAATATCTCATACACAA | |
| TTTCCTGTTAATACGTTTTAAACAAAACCTCCCTTAAGGAATTATTTCAATTAAGTATAAATATTCAAC | |
| AAGAACAGTTCTGATAAAGAATCCCCAGGTAGTTCAAATGCTAATGTGCACTGACCGAAGGAAAAGA | |
| AAAATAACAATTTTTGTTTGTTTTAACATGTTTATTACAACAGATACAATTCACATCTGACTAGCTCTG | |
| TTTCCTCTTTCCCCTCCCACAACCACGTTCATTGGGCCATTTCCTTATATTTGAGCAATCAATGTACTTT | |
| GAGCACACATGCCTGTCCAGCAGTACTTTTAAGTCCATTCTTACAGGGTGCAAATGGATTTCAATAATT | |
| TATACAAACAAGTAGGTTATGCTCAATCACTGCAATTTTAAGCTACTGTACACAGGAATGAAAAGGTT | |
| ATAGAAAAGTGCCATAGCAACAGTGCCTTAAGAAAGGAGATAAAGAGGAGCCTTAAAAAAATGGATA | |
| AAATCAGAATTTCAGAAGGAAATGGAAACACACGGGAAATGAAAAACATTTCTCTGCAAAACAAATG | |
| GAGAAGCACTGCTCTTGATCAGGTGCAAGTGTGGAAACAGTTGTTTCATATTTTGTACACTGCCCATAT | |
| GGTTCAAAATCGTATCCTTAGACACAAATCGCCTGGCGCTTGCACTGAATTTTTGAAAATGCAAGATTT | |
| CTGAATGATAAATTAACCCCCCCAATTTTTTTTTTTTTTTTTTTAAAAAAGCTAATTTTGCAGACAGGTT | |
| TACATGTAAAAGGCTAGGTATTTAGCCACCTCAGCATTGATTAGTTTTGGATGTCTAAGCTCTGTTACA | |
| CATGGCTTCCCATGGCTTCACTCTACAAAACATATTTACAACGTGAAGGATACATCTACAAGAAATCT | |
| ACATTTCAAGGGTTTTACAAATCAATCTTGTATCTTTCCCCTGAATTGACTCTCACAGACCCCGTCCCCT | |
| TGTCATTTCCTTTGCCCAGCTTAACGGTCCAAAGTCTACTTAAATGCAGCTCAAAAATGTTAAGATTGG | |
| GCACAACAGAATTTTACAGTTTTCCTGACTTCTTCAAAACCATGTGCAATTATTCACATCTTTCACACC | |
| ATGAAGGAATTCTGATTTTTTAGCTTTTCAAGTTCCTTAATTTGTTGTCTCAAAAATAGTATCCTTTGCT | |
| CTCGCAAAGTTGCTTGAATTTCACATACTCGGACTAAAGATCTTAAAUTTTTAATTCAGTACAAATTT | |
| CGGACATGTGAATAGTTCCCATGTTATGCTGCTGCTTTCCTTCGCGTAATCTTGATGCCTGCTTCTCTGC | |
| ATGATCTGCAGGCCATCCTTGAACGTGTTTTATGAGATTTTCACTGAAGTGTGCTGCTAGTGCAGGCGT | |
| TAATGAACACGTGGATCGGGCAGAAGAATTCTGTGGTACAACTGTTGCATTTGATGCATTACTACTATT | |
| AGAAGTATGATTGGGACCAGGTGATGGACTTCTCTGGCTACTTGAGCGCTGAAGACTTCGAGGAGAGA | |
| CAGGTTCATGACCTTGGCGCTTGTCAGCAGHACAGGCTGCTGTGTGGCTGACTGTGACACTGGTCCTT | |
| GCTTAACTACTGGAGTACTAACCTTTGGCTGTGATGAAACAGGAGGAGTACTGATCAAAGGTTTGATA | |
| GGGACTGTGTTAGTTTGAGGTGTGCTTATTCTTGGAGAAACATATGATCTTGGGGATGACGCATCAGA | |
| TGTTAAAGACATCGGAGACTGATTAGATGGCTGGGCTTGTGTAGAGAGCTGAGCAGCTTGAGAAATCA | |
| GAGACGTTATGTTGAAAGCAGATGGTCCAGCAGTAAGAAACTTATGAATTATAGACTGCAGTGAGGCT | |
| TGTGTCACAGCTGCTGTAAGAACTTCATTTATTTTAGATATGTCCACATTAGAATTATTAAGCTGCAGC | |
| GTGGCTTGCAAAGCAGGAAGCAATTGTCTAAGAAGATTTGGGTCCTGAAGTAAGGGAGGTATTGGCG | |
| ACTGTGGAACAGGAGAAACAGGGACCGCTGAAGCAGATGTTGGAGGTGCAGATGTGGGGTTCAGTCC | |
| AGAGGCAGAAGACGTGGAAGGAGTTGTGCAAGAATGTGATACGGGTTTGTCTCCTGATGTAGATTCTT | |
| TTCTTTCTGTTTTCTGTGCAGGGACAGAAGATGTGGGTGTAGGCAGTTTTGATAAAGTAGATGCTCCAT | |
| TAGCATCAAATGATTTCTTTGGCTGGTGATCAGACTGTAGCGTAAATGGACTAGAAGGAACAGTGCTT | |
| GGGGTAGCAGTTGGATGAACCACTGGTTTGATGGGGTGCTGTACTGGCGTAGAACTACTGTGAGTCTC | |
| TGCTCTTGGCAGTCTGTAGTCTCTGTCATTGTGTCTGCTTGTTTGAGACAAAATATTCTGTGGGAGCAA | |
| ACTACTGGCATCACTGGAATGCTTGTCTTCCATTCCACTGGCAAACCCACTAGTGGCTGTTGCTTGCAT | |
| CACCTCTCTTCTGTAATCCCTATCTTTTGGGAAGCTGTTGACTGCCATCTTGTTTGCTTCTTTTTGTCTCT | |
| GTTCTCTTTCAAGCCACTCTTTTGGTTTTTCCCATTGTGAAACTTCTGTTCGACAATTGTAGTAGTACTT | |
| TTTCCCAGAAGAGCTAATATGCTCAGACCAGTCATCTGCAGAATCATAAGGTGCATCTGAAGTTTTGCT | |
| TGGGTTATTGCTTGGATTAGAAGAATGTGAATTTGAACTATGAAGAGCACTGTGGTTGTGTGAATTTTC | |
| TTGTGGAGAGTAACTGGTCCCACCATCCCTCTCTCTAACTCTGTGAGTATGCACATTTTTGGCCTTACT | |
| GTGACCTGTGCTGTCACTGTATTTGTTTTCAGGACTATCAGATCTCCGCAACATTTTATTTGGTGGTGA | |
| AGGATCTCCGGCGTCTCGCATCTTTTCATGTCTGTGATCACCGCTACTGGGGTGACTCTTCGATGAATA | |
| CTTAAGTGCCTGGTAAGGCTGCGAGTCCCCCCTCCGGTCGTGACAGCCATCACTGAGTCTCTGCTGTTT | |
| CCTCGCATACATTACCATCAATGCCCGGCCTGTGAGTGTGTGTCGGGGAGGGGGGAGAGCGGCCGCGA | |
| GGGGGGAGAGCGGCCGCCCCCGCGGGAGACCTACCGAGCGCCCCAGCGCCGAGCCCCGCACGCACAC | |
| GGCACAAACCCGGAGCCTCCGGAAGGGCCGGAGAGGGCGAAATGCCGCGAAGCCGGGCCCCCGCTCT | |
| CCAACAACACTGCGTTCCCGCCCGACCGCCCGCGCGGCCGC | |
| KEANOX17 | |
| KEANOX17 is a novel 279 bp gene fragment. The nucleic acid has the | |
| following sequence: | |
| ACTAGTCCCACGTGCAGTTCGGTTGCTCCGAATGGGATTCGGGTGCGAGGGGCTGACTGG | (SEQ ID NO:17). |
| AGATGTGGGGTCTGGGTGAGTGGGGTATTTGAGAACCTGAAGCTCACAAGGCTTTGTCTG | |
| GTTTGAGGTACGTTTTGCCCTTTCTCCCGCATTCGGCTCTGCATCGAATCTGACCGGCCG | |
| CGCCTACTCTCGGAACGGCCGAGGAGGATGGGTCTCCCGTCTCCACACGCGCGGTGTCTG | |
| CGCCGACTGCAGCCCTCCTGGACGGGAGCCTCTGGGCCC | |
| KEANOX18 | |
| KEANOX18 is a novel 573 bp gene fragment. The nucleic acid has the | |
| following sequence: | |
| GCTCTGTTCTCCCANACTTGAAAGGCTCCTAGGGGATTTCCAGCACCCACCAANTTGGGCTGGGAAGA | (SEQ ID NO:18). |
| CAGGTTATTAAACAGTCTCTCCGGATACTGCACTGGGGCATTCAATGCCTCACAAGGACAGCGGTCCA | |
| GGTAAGTCAGATTCCAGCCGGGCTCTTCCTNTGTCCCATGTGATATCCGAAAGCCCCCGCCTCACTCTG | |
| AGACTTGGTTCCCCAACTTTGATGCAGTCGGGGAGGTCAGCCCTTGGTCCAGCCACTCCCCACAGTCAT | |
| GGTGGCTGAAAGCTGGTGTGCAACCTTGTTCCTCGAGGGGTCCCTGTGCCGTTCAGCAAGTCATGCAG | |
| CGCGGCCTCATGTCCCCCAAGAGTGGGUTGGATCCTCCTCAGAGTACAGGGGTGGCGGGCGGTAGCG | |
| GAACTCTTGOATGTAGGCGAAGAACGGGCCTTCAAGGCTCATGTCGGGGTCCTGCGGAAGCGGNGTAT | |
| GGCCTTCCCAAGGCTGGCCTCCTCAGTGTCGGCTACCACCTCCGAGTACTCAGGAGGAGCCTCAGGCC | |
| GCTCCGGCAAGGCCCCAGGCTCCAGTC | |
| KEANOX19 | |
| KEANOX19 is a novel 643 bp gene fragment. The nucleic acid has the | |
| following sequence: | |
| GCGGCCGCCCGCCTTGGCCCGTCTCTGGAGTGCTGGGCAGCCGGGTCTGCGGGCCCCTTTACAGCACA | (SEQ ID NO:19). |
| TCGCCGGCCGGCCCAGGTAGGGCGGCCTCTCTCCCTCGCAAGGGGGCCCAGCTGGAGCTGGAGGAGA | |
| TGCTGGTCCCCAGGAAGATGTCCGTCAGCCCCCTGGAGAGCTGGCTCACGGCCCGCTGCTTCCTGCCC | |
| AGACTGGATACCGGGACCGCAGGGACTGTGGCTCCACCGCAATCCTACCAGTGTCCGCCCAGCCAGAT | |
| AGGGGAAGGGGCCGAGCAGGGGGATGAAGGCGTCGCGGATGCGCCTCAAATTCAGTGCAAAAACGTG | |
| CTGAAGATCCGCCGGCGGAAGATGAACCACCACAAGTACCGGAAGCTGGTGAAGAAGACGCGGTTCC | |
| TGCGGAGGAAGGTCCAGGAGGGACGCCTGAGACGCAAGCAGATCAAGTTCGAGAAAGACCTGAGGC | |
| GCATCTGGCTGAAGGCGGGGCTAAAGGAAGCCCCCGAAGGCTGGCAGACCCCCAAGATCTACCTGCG | |
| GGGCAAATGAGTCTGCGCCGCCCTTCCCGCCCGTTGCTGCTGTGATCCGTAGTAATAAATTCTCAGAG | |
| GACTCAGAAAAAAAAAAAAAAAACGTGCTGCCGAAAT | |
| KEANQX20 | |
| KEANOX20 is a novel 1513 bp gene fragment. The nucleic acid has the | |
| following sequence: | |
| CCCGGGTTTTTTTTTTTTTTTTTTTTTTTTTTTTTTTTTTTTTTTTTTTTTTTTTTTTTTTCTTTTTTTTTTTTT | (SEQ ID NO:20). |
| TTTTTTCGTGACACAAACATGCATTCGTTTTATTCACAAAACAGCCTGGTTTCCTAAAACAATACAAAC | |
| AGCATGTTCATCAGCAGGAAGCTGGCCGTGGGCAGGGGGGCCCAGGCCGCAGGGGGCCCCCTGGGGA | |
| CCCACCCCCACCAGCAGGGGACCACGCAAAGAAGCCCTTTCTTCTGTTGCCATTAGCAAGGCCAGAAA | |
| AAAAGGACTTATTTTCTCTAAGAAAAGTAGCCAGGATTGGAAATATCGAGATGGGGCTCCCCAGATCG | |
| CAATGGATAAACGAACAGAACTTTCATTTTTTTTTCCAAAATCACTGTTGGGGTGGGGGATCCCAGTCT | |
| CGGGACTGTGGGTGCAGCTCAGCTGGCCTGGTGCTCCCTCCCCTTCTGTGAGCTCCCTGCTGGCAGCTC | |
| TGCACGCGTGGATGGATACAGAGCGGCTTCTACACGGCGCCATCAACATTCTCTTTATGAACGTGAGT | |
| GGATTCTCCAGGCAAACTATGCACTATTTCATGGTCGGAAAGAATCAAAGGAAGTTTAAATGAGGGTG | |
| GAGTTAAACTGTGCTAAATTACAGTAGTGCTTATTAGTAACTAGATTTCAAAAGGTTACAGAAAATTT | |
| ACATTCTCTACACAAAAACTGCATCTCCTGCACAGACAACATCGACATCGACACAGGAAGGAAACTGA | |
| TTGTCCATTCTTTGCCCAGGAAGTCTCGGTACTTTATAGATTCGTCTTTACCTCTTTTTTGTTGTTGTTCT | |
| TCCGAAAAAGCAGTTTAAATTTTTTTCTTTTTCTTTTTATGCTAGGGACGTGGAGATGTTAAAACGACA | |
| ACAAAAAATATATATATAAAAACAGGAATGAAATCTGTGAGAGAATATTTTTGGTTCTAAAGACGGGT | |
| GCATCCGTTTGTCTTCGCCCGAATCCCTTGCTGGAGACCACACGAGCAGTGACATTGCACGGAGAGGG | |
| CAGCTTTGGGTTCCGCCGCCGTCACTGAAACCACCGGAAGGCGGCTCCCGTCGGAAGCATCACCTTCT | |
| GGCGGGGGCGGGAGACAAAAACAAGGAGAGAGTTCAGTCAGGGTCCGGGTAGGCCTGGAGCAAAGA | |
| CGTTCCCCCATGGATGGCCTCATGGCGGAGGGACAGGCATGGAGGAAGAGCAGCCACCCCTCGGAGC | |
| TCCTGCCACCCCAGCCAGGCCAAAGGAGGCGCTTGACCTGCTGGGCTGCTGGAAGGCCCCGAGGACTC | |
| TGAGGCCTCCTGAGCCACTGTGCCCAGGAACCCTCCTTCCAGAGAAGAAGTCCCTTCCCCTGGCCCAA | |
| AGGCAACANCGCCAGGTGGCTGTGNCATGTNTCTGTAAATGAGCGTGTGAGCACGTNCNANGGGTGT | |
| GCATATGGNTGTGTGCCTCGTGCTATGCTGAGCTGTGTGGTATGGANCTGTGGGAGGTGGGTATTTGA | |
| GCTATTTC | |
| KEANOX21 | |
| KEANOX21 is a novel 1432 bp gene fragment. The nucleic acid has the | |
| following sequence: | |
| GCTCGACTTTTTTTTTTTTTTTTTTTTTTTTTTTTTTTTTTTAGGGTTCAGTTCCAGCTGATTTTAT | (SEQ ID NO:21). |
| TTCCTTCTCAAAAAAAGTTATTTACAGAAGGTATATATCAACAATCTGACAGGCAGTGAACTT | |
| GACATGATTAGCTGGCATGATTTTTTCTTTTTTTTCCCCCAAACATTGTTTTTGTGGCCTTGAAT | |
| TTTAAGACAAATATTCTACACGGCATATTGCACAGGATGGATGGCAAAAAAAAGTTTAAAAA | |
| CAAAAACCCTTAACGGAACTGCCTTAAAAAGGCAGACGTCCTAGTGCCTGTCATGTTATATTA | |
| AACATACATACACACAATCTTTTTGCTTATTATAATACAGAGTTAAATGTACAAAGATGTTTTC | |
| CACTTTTTTCAATTTTTAAACACAACAGCTATAAACCTGAACACATATGCTATCATCATGCCAT | |
| AAGACTAAAACAATTATATTTAGCGACAAGTAGAAAGGATTAAATAGTCAAATACAAGAATG | |
| AAAAACGCAGTACATAGTGTCGCGAACTCAAATCGGCATTTAGATAGATCCAGTGGTTTAAAC | |
| GGCACGTTTTTGCTTATAAAAAAAGTGCAAAAAAGATGTGGTTTACAAGTTAAAGCTACAGA | |
| ATCCCTTTTTGCTGTAATTGCACGAGTTTTAAAGCCTCTGGACAGAGCAGTATTTCGTTTAAAA | |
| CTTTGTTTTTCTTAAAAGCTTACAGTGTTTGGCTAATTCTCCTCCCCTTTTTACAAGACGGGGG | |
| CCGGAGGGTGGACACTGGTGGCAGGTTAAGGGATACTGTCACTTTAAGAAGCCTGCAGATTG | |
| AAGTGTAAACATGGAGAAATTAGGGGCTGATTTTTTAAACTGTGTGAGATATTAACCAGCCGC | |
| CCTGTTATAAAATCAGGAAATCCAAACAGCGATTTACACCGATTAACACCCCCTTTATATATT | |
| TTTTACAAAAATACACTGAGAAAATAATCAAACGTTTTCATCTCTCTTGTCTTTTTTTGTTTTTT | |
| AAAAGTGTCAAAAGTCTACATTTAAATATAAAAAATTAAAAGTTAAAACTCTAGCCCTTCAGT | |
| GAAGGAGACGTAAAATGGCGTGGGTAACAACAACTACCAAAAAAAAAAAAAAAAAAAAAGA | |
| AAAAAAGGAAAAGGAAGGAATAAAGAAATAAAGGAAGTAAAAAGAAAGGAAAGAAAAAAA | |
| GGGACAAAAGAAAAAATATGTTTGGCCCAGTATAAATACGTCCCACATATAAAATGGCATCT | |
| GATTACATTTACAAGGAAAAAGAAATACGAGGATGGAGCATCGGTGAAGGAAAAAAACACG | |
| TCTTCTCATTTACACCTATAAGGAATAAACACACACACTGAGAAAAAATTTGGTCCTGAATTG | |
| TTTTTTTAAAGTCCAGCACAGATTTGAGTTGCGTTTGAATCCTTTA | |
| KEANOX22 | |
| KEANOX22 is a novel 860 bp gene fragment. The nucleic acid has the | |
| following sequence: | |
| AGCGGCCGCCACCAGCGTCACCAGCAGCACCGCCAGCTTAGCCTTCATGTGCAGGCTCGAGCCCAGGT | (SEQ ID NO:22). |
| ACACGGTGAAGATGGCCACAGCCTGCCACCAGTGGTAGATGGTGAAGATGAAGTCCTGTCTCTCCTTG | |
| TCTTCGTACAAGATTCCCAGGAGTGTGCTGAGTCCAGTCTTGTTCAGGGCACTGCCCACACCCCAAAG | |
| GGCAGCTGCCACATAGAGGATCCAGCTGTGTTGCAGGACCCGAGGCACAGGGGCCCAGAAAAAGAGG | |
| ATGAAGGTGAGCAGCAGGTGCACCCCTGCTCCGGCCACCAGGGGCACCGGGCGTGGCAGCCACAGGC | |
| CCAGCAGGCCCAGGAGTGAGGCGGCTGAGGCGCCCAGGCTGTAAGCCACGAGGAGGTAAGCCAGCCG | |
| CTCCAGCCCCACCGAGCACACGCCATAGCCCAAGGCGATACCAGTGCAGGCAAAGAGCACCTCGAAG | |
| CCGCTGTAGATAAAGAAAGGCACGAGGTGGCGCAGGCGGTAGTCACGCACGTGCTTGAAGGGCAGCT | |
| GGAAGATGTTGCCCCAGCCCACGCTGCGCAGATCGATCTCCTCCGTGGGCCGGTAAGCGGCTCCGCAC | |
| AAACCCAGCACCAGCAGCATGGCCAGGAAGGCCACTGCCATGAGCACGCTCTCCACCACAATGAGGT | |
| TTCCCGCTCCGCGGGAGCGTCCGCAGAAGCGTCTTGTTGAAGCCGCTGAGGATCCCGTGGCTGTTGGT | |
| GCCGCAGCTCTGCACATTGTACAGCGTGTGGTTCAGGTCATACAGGTAGTGGTTCAGGAAATAAATCA | |
| TGGGCAGCTGGGCGCAGGCGAAGCTCAGATGGAAGAAGCTGTAGAAGATG | |
| KEANOX23 | |
| KEANOX23 is a novel 780 bp gene fragment. The nucleic acid has the | |
| following sequence: | |
| TTTTTTTTTTTTTTTTTTTAGGTTTGTTAACATTAACACATGCTTTATTCAAGAGATAATATTCAAAGAG | (SEQ ID NO:23). |
| TTAAATCCTAAGAGTTATCCACCCTACAGTAAAAAGGGGAAGTGGGTACCACTTATGACATGTACATA | |
| AATTCCACTTTTATATTTCTGAATAAAGCTGCAATTGCTTCTTTGATAGAGCCATTTCTTAAAACTTTTG | |
| CTAATAAGGCTATGTGAACTGTGTTCAGAAACTTTGACAACATGCACACTCACTCCTCTCAAAGTCAGT | |
| ACCCAGGATTTTCACTCCAGGCTGAGTACCTATTAAGTAACTAGGACTTCAGACTGCATGTTACTATAT | |
| GAATTCAATTTGACTCACCTCCAGTATGTTTATCTACCACAACTATTGTTTAAAATAATCAGATGAATG | |
| TTTATCATAACTFTTATAACTCCCCCAAATTATACTTCAGTATHAACATGGTAGTTTCAAAAAATAAAT | |
| ATTCAAGGCCCCAGTTTTAAATTTTCCTCCAATGTAATCCACAAAGTTGATAATACATGTTAGGAGCAC | |
| TACTCTAAAATAAATGTAGCTTTCTTTTAAAATTACTTGTTGCAATAGAGATCACATTCTATTCCAAAT | |
| TCTGATAAGGAATCTGTTTTTTTGGTAATACAACAAAAATTGGGGATGAGTCAGTCCTTGGAAAATCTT | |
| AATAGTAGTCACTGAACGAAGTCAATCATTCCGAGAGCNCACAACACTCAGAAATTACTGCTAGCATT | |
| GACAAGTTCTTGTGTTTTTTG | |
| KEANOX24 | |
| KEANOX24 is a novel 367 bp gene fragment. The nucleic acid has the | |
| following sequence: | |
| TACCCCATTGCCGGACAAAGCTTTGTCAACTTGCAGTGCTTCTGGCTCCCGAAGAAGCACTGTCTGAA | (SEQ ID NO:24). |
| ACCACTATTTCTTTTTCCCTCATCCCACTCTAAAAGGTAGTTGGTGATTTTTGAACCGTTGTCAATTGGT | |
| GCCTTCCACTGCAGGGTTAGTGAACTTTTGCTCCTATGTGCCAGCTTAGGGGGGAAAGGACACTCGGG | |
| TGCACAGCTGTGGGTGGTGAAGCTAACAGGCTCGGAGCAGGATCCCTTTACGGAATTGTACATGGCAT | |
| ACACCCTCACATGATAATCTGTTGCTGGTCTAAGATCTTTCAGGTTACATTCTAATTCTTCTCCACTGTA | |
| AATTATCTTGTATTTTCTCGTGC | |
| KEANOX25 | |
| KEANOX25 is a novel 854 bp gene fragment. The nucleic acid has the | |
| following sequence: | |
| ACATGTTGCCCAGGCTGAGATTATTTTTATTTAAAAAAAAAAGATTGCAATATGAACTCTCTCCCTTCC | (SEQ ID NO:25). |
| TAAACAGATAATGCAGACTTTTTTTTCCTTTCAAATGAAGAGGTTTTATTGGAAGTTGCATAGAGAACA | |
| CAGTCAGTCACGAGAGCAGAAGGAGCAGGTTCCAGGATTCTGGGGAGATAAGGGTCCAAGCTGAGAA | |
| CTCCCCTGAGAAGTCTGTATGCAGACAGCCCCGGGTGATCCTGGCTGTCCAAGGGCTGAGGGCGGGTT | |
| GGGAAGGGCCCTCATCACCCCAGGGGCCAGCTCCCCACATAGCAGCTGAGTAGCACCCAGAGAACCC | |
| GGATCTTCACTCAAGGTGGCTGGCAGCAGAGCCACAGCCCAGGCCACCAAAGCCACCAGAGCCCCTG | |
| TGTGGCAAGGATCATGATGCGAGGGTGGTGAGTTGGTTGCTGTTCTCAGCAGGATCCTGGGTGTAAAT | |
| GGCCATGCCTGTCCAGCACGGCTTATGGGGCTGAGCATGTGAGAACTTGTGTGGCCTGTGCCTGTGCA | |
| AACGCGTGTGCCCTGGGTATCACACCCAGAGCCCTGCTGTGTGAAGAAGCCTCAGTCATCACGTGTGA | |
| GCTCACAGTGGGCTGGTCATGGGGCCCCTTCCACATGGTGCACTGCTGCTGCAGGAATTGCTGTGTCTG | |
| CAAATTCTGGACATACCAGACCCTAAATAGAATGCTGGGAGCAGGCAGGTTACAGAAGCCTCTGAGT | |
| GCGGCAGCTCTATGGGAACTGGGGTGCTTCACTTACAAGTATTTGAGGTATTACAATGTATCCAATTTT | |
| TTTTTTTTTTTTTTTTTGAAACGGGGGCTTCCTCTCCC | |

Screening for Integrin Modulating Agents

In one aspect, the invention provides a method of identifying integrin modulating agents. By integrin modulating agent is meant that the agent modulates (i. e., increases or decreases) integrin levels or activity. These agents include for example, integrin activators, inhibitors, endogenous and exogenous ligands, and integrin-binding proteins. Integrin ligands include for example extracellular matrix proteins, such fibronectin, collagen and laminin, and other cell surface proteins. Integrin inhibitors include small molecules, e.g. cyclic peptides; peptidomimetics; antibodies, e.g. LM609, an alphavbeta3-disrupting antibody; and tight-binding inhibitors, eg. BIO-1211, which inhibits integrin-mediated inflammation. Integrin-binding proteins include for example intracellular, extracellular and plasma membrane-associated proteins involved in cell shape, motility, proliferation, differentiation, and adhesion.

The integrin modulating agent can be identified by providing a cell population that includes cells capable of expressing one or more nucleic acid sequences homologous to those listed in Tables 1-3 as KEANOX 1-260. Preferably, the cell population includes cells capable of expressing one or more nucleic acids sequences homologous to KEANOX 1-260. By “capable of expressing” is meant that the gene is present in an intact form in the cell and can be expressed. The sequences need not be identical to sequences including KEANOX 1-260, as long as the sequence is sufficiently similar that specific hybridization can be detected. Preferably, the cell includes sequences that are identical, or nearly identical to those identifying the KEANOX nucleic acids shown in Tables 1-3.

Expression of one, some, or all of the KEANOX sequences is then detected, if present, and, preferably, measured. Using sequence information provided by the database entries for the known sequences, or the sequence information for the newly described sequences, expression of the KEANOX sequences can be detected (if present) and measured using techniques well known to one of ordinary skill in the art. For example, sequences within the sequence database entries corresponding to KEANOX sequences, or within the sequences disclosed herein, can be used to construct probes for detecting KEANOX RNA sequences in, e.g., northern blot hybridization analyses or methods which specifically, and, preferably, quantitatively amplify specific nucleic acid sequences. As another example, the sequences can be used to construct primers for specifically amplifying the KEANOX sequences in, e.g., amplification-based detection methods such as reverse-transcription based polymerase chain reaction. When alterations in gene expression are associated with gene amplification or deletion, sequence comparisons in test and reference populations can be made by comparing relative amounts of the examined DNA sequences in the test and reference cell populations.

Expression can be also measured at the protein level, i.e., by measuring the levels of polypeptides encoded by the gene products described herein. Such methods are well known in the art and include, e.g., immunoassays based on antibodies to proteins encoded by the genes.

Expression level of one or more of the KEANOX sequences in the test cell population is then compared to expression levels of the sequences in one or more cells from a reference cell population. Expression of sequences in test and control populations of cells can be compared using any art-recognized method for comparing expression of nucleic acid sequences. For example, expression can be compared using GENECALLING® methods as described in U.S. Pat. No. 5,871,697 and in Shimkets et al., Nat. Biotechnol. 17: 798-803.

In various embodiments, the expression of 2, 3, 4, 5, 6, 7, 8, 9, 10, 15, 20, 25, 35, 40, 50, 100, 150, 200 or all of the sequences represented by KEANOX 1-260 are measured. If desired, expression of these sequences can be measured along with other sequences whose expression is known to be altered according to one of the herein described parameters or conditions.

The KEANOX nucleic acids and encoded polypeptides can be identified using the information provide above. In some embodiments, the KEANOX nucleic acids and polypeptide correspond to nucleic acids or polypeptides which include the various sequences (referenced by SEQ ID NOs) disclosed for each KEANOX polypeptide.

Expression of the nucleic acid sequences in the test cell population is then compared to the expression of the nucleic acid sequences in a reference cell population, which is a cell population that has not been exposed to the test agent, or, in some embodiments, a cell population exposed the test agent. Comparison can be performed on test and reference samples measured concurrently or at temporally distinct times. An example of the latter is the use of compiled expression information, e.g, a sequence database, which assembles information about expression levels of known sequences following administration of various agents. For example, alteration of expression levels following administration of test agent can be compared to the expression changes observed in the nucleic acid sequences following administration of a control agent, such as fibronectin, laminin or Type I collagen.

An alteration (i.e., increase or decrease) in expression of the nucleic acid sequence in the test cell population compared to the expression of the nucleic acid sequence in the reference cell population that has not been exposed to the test agent indicates the test agent is a integrin modulating agent.

The reference cell population includes one or more cells for which the compared parameter is known. The compared parameter can be, e.g., integrin modulating agent expression status. By “integrin modulating agent expression status” is meant that it is known whether the reference cell has had contact with an integrin modulating agent. Example of an integrin modulating agent are extracellular matrix component such as fibronectin, laminin or collagen. Whether or not comparison of the gene expression profile in the test cell population to the reference cell population reveals the presence, or degree, of the measured parameter depends on the composition of the reference cell population. For example, if the reference cell population is composed of cells that have not been treated with a known integrin modulating agent, a similar gene expression level in the test cell population and a reference cell population indicates the test agent is not an integrin modulating agent. Conversely, if the reference cell population is made up of cells that have been treated with an integrin modulating agent, a similar gene expression profile between the test cell population and the reference cell population indicates the test agent is an integrin modulating agent.

In various embodiments, a KEANOX sequence in a test cell population is considered comparable in expression level to the expression level of the KEANOX sequence if its expression level varies within a factor of 2.0, 1.5, or 1.0 fold to the level of the KEANOX transcript in the reference cell population. In various embodiments, a KEANOX sequence in a test cell population can be considered altered in levels of expression if its expression level varies from the reference cell population by more than 1.0, 1.5, 2.0 or more fold from the expression level of the corresponding KEANOX sequence in the reference cell population.

If desired, comparison of differentially expressed sequences between a test cell population and a reference cell population can be done with respect to a control nucleic acid whose expression is independent of the parameter or condition being measured. Expression levels of the control nucleic acid in the test and reference nucleic acid can be used to normalize signal levels in the compared populations.

In some embodiments, the test cell population is compared to multiple reference cell populations. Each of the multiple reference populations may differ in the known parameter. Thus, a test cell population may be compared to a first reference cell population known to have been exposed to an integrin modulating agent, as well as a second reference population known to have not been exposed to an integrin modulating agent.

The test cell population that is exposed to, i.e., contacted with, the test integrin modulating agent can be any number of cells, i.e., one or more cells, and can be provided in vitro, in vivo, or ex vivo.

In other embodiments, the test cell population can be divided into two or more subpopulations. The subpopulations can be created by dividing the first population of cells to create as identical a subpopulation as possible. This will be suitable, in, for example, in vitro or ex vivo screening methods. In some embodiments, various sub populations can be exposed to a control agent, and/or a test agent, multiple test agents, or, e.g., varying dosages of one or multiple test agents administered together, or in various combinations.

Preferably, cells in the reference cell population are derived from a tissue type as similar as possible to test cell, e.g., blood cell, immune cell or monocyte. In some embodiments, the control cell is derived from the same subject as the test cell, e.g., from a region proximal to the region of origin of the test cell. In other embodiments, the reference cell population is derived from a plurality of cells. For example, the reference cell population can be a database of expression patterns from previously tested cells for which one of the herein-described parameters or conditions (integrin modulating agent expression status is known).

The test agent can be a compound or composition (e.g., protein, nucleic acid, small molecule, or antibody) not previously described or can be a previously known compound but which is not known to be an integrin modulating agent.

The invention also includes an integrin modulating agent identified according to this screening method, and a pharmaceutical composition which includes the integrin modulating agent.

The subject is preferably a mammal. The mammal can be, e.g, a human, non-human primate, mouse, rat, dog, cat, horse, or cow.

KEANOX Nucleic Acids

Also provided in the invention are novel nucleic acid comprising a nucleic acid sequence selected from the group consisting of KEANOX: 1-25, or its complement, as well as vectors and cells including these nucleic acids.

Thus, one aspect of the invention pertains to isolated KEANOX nucleic acid molecules that encode KEANOX proteins or biologically active portions thereof. Also included are nucleic acid fragments sufficient for use as hybridization probes to identify KEANOX-encoding nucleic acids (e.g., KEANOX mRNA) and fragments for use as polymerase chain reaction (PCR) primers for the amplification or mutation of KEANOX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but preferably is double-stranded DNA.

“Probes” refer to nucleic acid sequences of variable length, preferably between at least about 10 nucleotides (nt) or as many as about, e.g., 6,000 nt, depending on use. Probes are used in the detection of identical, similar, or complementary nucleic acid sequences. Longer length probes are usually obtained from a natural or recombinant source, are highly specific and much slower to hybridize than oligomers. Probes may be single- or double-stranded and designed to have specificity in PCR, membrane-based hybridization technologies, or ELISA-like technologies.

An “isolated” nucleic acid molecule is one that is separated from other nucleic acid molecules which are present in the natural source of the nucleic acid. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA or RNA molecules. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated KEANOX nucleic acid molecule can contain less than about 50 kb, 25 kb, 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the present invention, e.g., a nucleic acid molecule having the nucleotide sequence of any of KEANOX: 1-25, or a complement of any of these nucleotide sequences, can be isolated using standard molecular biology techniques and the sequence information provided herein. Using all or a portion of these nucleic acid sequences as a hybridization probe, KEANOX nucleic acid sequences can be isolated using standard hybridization and cloning techniques (e.g., as described in Sambrook et al., eds., MOLECULAR CLONING: A LABORATORY MANUAL 2^{nd} Ed., Cold Spring Harbor Laboratory Press, Cold Spring Harbor, N.Y., 1989; and Ausubel, et al., eds., CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to KEANOX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having at least about 10 nt and as many as 50 nt, preferably about 15 nt to 30 nt. They may be chemically synthesized and may be used as probes.

In another embodiment, an isolated nucleic acid molecule of the invention comprises a nucleic acid molecule that is a complement of the nucleotide sequence shown in KEANOX: 1-25. In another embodiment, an isolated nucleic acid molecule of the invention comprises a nucleic acid molecule that is a complement of the nucleotide sequence shown in any of these sequences, or a portion of any of these nucleotide sequences. A nucleic acid molecule that is complementary to the nucleotide sequence shown in KEANOX: 1-25 is one that is sufficiently complementary to the nucleotide sequence shown, such that it can hydrogen bond with little or no mismatches to the nucleotide sequences shown, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, Von der Waals, hydrophobic interactions, etc. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Moreover, the nucleic acid molecule of the invention can comprise only a portion of the nucleic acid sequence of KEANOX: 1-25 e.g., a fragment that can be used as a probe or primer or a fragment encoding a biologically active portion of KEANOX. Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 45%, 50%, 70%, 80%, 95%, 98%, or even 99% identity (with a preferred identity of 80-99%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below. An exemplary program is the Gap program (Wisconsin Sequence Analysis Package, Version 8 for UNIX, Genetics Computer Group, University Research Park, Madison, Wis.) using the default settings, which uses the algorithm of Smith and Waterman (Adv. Appl. Math., 1981, 2: 482-489, which in incorporated herein by reference in its entirety).

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of a KEANOX polypeptide. Isoforms can be expressed in different tissues of the same organism as a result of, for example, alternative splicing of RNA. Alternatively, isoforms can be encoded by different genes. In the present invention, homologous nucleotide sequences include nucleotide sequences encoding for a KEANOX polypeptide of species other than humans, including, but not limited to, mammals, and thus can include, e.g., mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the nucleotide sequence encoding a human KEANOX protein. Homologous nucleic acid sequences include those nucleic acid sequences that encode conservative amino acid substitutions (see below) in a KEANOX polypeptide, as well as a polypeptide having a KEANOX activity. A homologous amino acid sequence does not encode the amino acid sequence of a human KEANOX polypeptide.

The nucleotide sequence determined from the cloning of human KEANOX genes allows for the generation of probes and primers designed for use in identifying and/or cloning KEANOX homologues in other cell types, e.g., from other tissues, as well as KEANOX homologues from other mammals. The probe/primer typically comprises a substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of a nucleic acid comprising a KEANOX sequence, or an anti-sense strand nucleotide sequence of a nucleic acid comprising a KEANOX sequence, or of a naturally occurring mutant of these sequences.

Probes based on human KEANOX nucleotide sequences can be used to detect transcripts or genomic sequences encoding the same or homologous proteins. In various embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a KEANOX protein, such as by measuring a level of a KEANOX-encoding nucleic acid in a sample of cells from a subject e.g., detecting KEANOX mRNA levels or determining whether a genomic KEANOX gene has been mutated or deleted.

“A polypeptide having a biologically active portion of KEANOX” refers to polypeptides exhibiting activity similar, but not necessarily identical to, an activity of a polypeptide of the present invention, including mature forms, as measured in a particular biological assay, with or without dose dependency. A nucleic acid fragment encoding a “biologically active portion of KEANOX” can be prepared by isolating a portion of KEANOX: 1-25, that encodes a polypeptide having a KEANOX biological activity, expressing the encoded portion of KEANOX protein (e.g., by recombinant expression in vitro) and assessing the activity of the encoded portion of KEANOX. For example, a nucleic acid fragment encoding a biologically active portion of a KEANOX polypeptide can optionally include an ATP-binding domain. In another embodiment, a nucleic acid fragment encoding a biologically active portion of KEANOX includes one or more regions.

KEANOX Variants

The invention further encompasses nucleic acid molecules that differ from the disclosed or referenced KEANOX nucleotide sequences due to degeneracy of the genetic code. These nucleic acids thus encode the same KEANOX protein as that encoded by nucleotide sequence comprising a KEANOX nucleic acid as shown in, e.g., KEANOX1-25.

It will be appreciated by those skilled in the art that DNA sequence polymorphisms that lead to changes in the amino acid sequences of a KEANOX polypeptide may exist within a population (e.g., the human population). Such genetic polymorphism in the KEANOX gene may exist among individuals within a population due to natural allelic variation. As used herein, the terms “gene” and “recombinant gene” refer to nucleic acid molecules comprising an open reading frame encoding a KEANOX protein, preferably a mammalian KEANOX protein. Such natural allelic variations can typically result in 1-5% variance in the nucleotide sequence of the KEANOX gene. Any and all such nucleotide variations and resulting amino acid polymorphisms in KEANOX that are the result of natural allelic variation and that do not alter the functional activity of KEANOX are intended to be within the scope of the invention.

Moreover, nucleic acid molecules encoding KEANOX proteins from other species, and thus that have a nucleotide sequence that differs from the human sequence of KEANOX1-25, are intended to be within the scope of the invention. Nucleic acid molecules corresponding to natural allelic variants and homologues of the KEANOX DNAs of the invention can be isolated based on their homology to the human KEANOX nucleic acids disclosed herein using the human cDNAs, or a portion thereof, as a hybridization probe according to standard hybridization techniques under stringent hybridization conditions. For example, a soluble human KEANOX DNA can be isolated based on its homology to human membrane-bound KEANOX. Likewise, a membrane-bound human KEANOX DNA can be isolated based on its homology to soluble human KEANOX.

Accordingly, in another embodiment, an isolated nucleic acid molecule of the invention is at least 6 nucleotides in length and hybridizes under stringent conditions to the nucleic acid molecule comprising the nucleotide sequence of KEANOX: 1-25. In another embodiment, the nucleic acid is at least 10, 25, 50, 100, 250 or 500 nucleotides in length. In another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding KEANOX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions is hybridization in a high salt buffer comprising 6X SSC, 50 mM Tris-HCl (pH 7.5), 1 mM EDTA, 0.02% PVP, 0.02% Ficoll, 0.02% BSA, and 500 mg/ml denatured salmon sperm DNA at 65° C. This hybridization is followed by one or more washes in 0.2X SSC, 0.01% BSA at 50° C. An isolated nucleic acid molecule of the invention that hybridizes under stringent conditions to the sequence of KEANOX: 1-25 corresponds to a naturally occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of KEANOX: 1-25 or fragments, analogs or derivatives thereof, under conditions of moderate stringency is provided. A non-limiting example of moderate stringency hybridization conditions are hybridization in 6X SSC, 5X Denhardt's solution, 0.5% SDS and 100 mg/ml denatured salmon sperm DNA at 55° C., followed by one or more washes in 1X SSC, 0.1% SDS at 37° C. Other conditions of moderate stringency that may be used are well known in the art. See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY.

In a third embodiment, a nucleic acid that is hybridizable to the nucleic acid molecule comprising the nucleotide sequence of KEANOX: 1-25 or fragments, analogs or derivatives thereof, under conditions of low stringency, is provided. A non-limiting example of low stringency hybridization conditions are hybridization in 35% formamide, 5X SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2X SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo et al., 1981, Proc Natl Acad Sci USA 78: 6789-6792.

Conservative Mutations

In addition to naturally-occurring allelic variants of the KEANOX sequence that may exist in the population, the skilled artisan will further appreciate that changes can be introduced into an KEANOX nucleic acid or directly into an KEANOX polypeptide sequence without altering the functional ability of the KEANOX protein. In some embodiments, the nucleotide sequence of KEANOX: 1-25 will be altered, thereby leading to changes in the amino acid sequence of the encoded KEANOX protein. For example, nucleotide substitutions that result in amino acid substitutions at various “non-essential” amino acid residues can be made in the sequence of KEANOX: 1-25. A “non-essential” amino acid residue is a residue that can be altered from the wild-type sequence of KEANOX without altering the biological activity, whereas an “essential” amino acid residue is required for biological activity. For example, amino acid residues that are conserved among the KEANOX proteins of the present invention, are predicted to be particularly unamenable to alteration.

In addition, amino acid residues that are conserved among family members of the KEANOX proteins of the present invention, are also predicted to be particularly unamenable to alteration. As such, these conserved domains are not likely to be amenable to mutation. Other amino acid residues, however, (e.g., those that are not conserved or only semi-conserved among members of the KEANOX proteins) may not be essential for activity and thus are likely to be amenable to alteration.

Another aspect of the invention pertains to nucleic acid molecules encoding KEANOX proteins that contain changes in amino acid residues that are not essential for activity. Such KEANOX proteins differ in amino acid sequence from the amino acid sequences of polypeptides encoded by nucleic acids containing KEANOX: 1-25, yet retain biological activity. In one embodiment, the isolated nucleic acid molecule comprises a nucleotide sequence encoding a protein, wherein the protein comprises an amino acid sequence at least about 45% homologous, more preferably 60%, and still more preferably at least about 70%, 80%, 90%, 95%, 98%, and most preferably at least about 99% homologous to the amino acid sequence of the amino acid sequences of polypeptides encoded by nucleic acids comprising KEANOX: 1-25.

An isolated nucleic acid molecule encoding a KEANOX protein homologous to can be created by introducing one or more nucleotide substitutions, additions or deletions into the nucleotide sequence of a nucleic acid comprising KEANOX: 1-25, such that one or more amino acid substitutions, additions or deletions are introduced into the encoded protein.

Mutations can be introduced into a nucleic acid comprising KEANOX: 1-25 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in KEANOX is replaced with another amino acid residue from the same side chain family. Alternatively, in another embodiment, mutations can be introduced randomly along all or part of a KEANOX coding sequence, such as by saturation mutagenesis, and the resultant mutants can be screened for KEANOX biological activity to identify mutants that retain activity. Following mutagenesis of the nucleic acids the encoded protein can be expressed by any recombinant technology known in the art and the activity of the protein can be determined.

In one embodiment, a mutant KEANOX protein can be assayed for (1) the ability to form protein:protein interactions with other KEANOX proteins, other cell-surface proteins, or biologically active portions thereof, (2) complex formation between a mutant KEANOX protein and a KEANOX ligand; (3) the ability of a mutant KEANOX protein to bind to an intracellular target protein or biologically active portion thereof; (e.g., avidin proteins); (4) the ability to bind ATP; or (5) the ability to specifically bind a KEANOX protein antibody. In other embodiment, the fragment of the complementary polynucleotide sequence described in claim 1 wherein the fragment of the complementary polynucleotide sequence hybridizes to the first sequence.

In other specific embodiments, the nucleic acid is RNA or DNA. The fragment or the fragment of the complementary polynucleotide sequence described in claim 38, wherein the fragment is between about 10 and about 100 nucleotides in length, e.g., between about 10 and about 90 nucleotides in length, or about 10 and about 75 nucleotides in length, about 10 and about 50 bases in length, about 10 and about 40 bases in length, or about 15 and about 30 bases in length.

Antisense

Another aspect of the invention pertains to isolated antisense nucleic acid molecules that are hybridizable to or complementary to the nucleic acid molecule comprising the nucleotide sequence of a KEANOX sequence or fragments, analogs or derivatives thereof. An “antisense” nucleic acid comprises a nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to at least about 10, 25, 50, 100, 250 or 500 nucleotides or an entire KEANOX coding strand, or to only a portion thereof. Nucleic acid molecules encoding fragments, homologs, derivatives and analogs of a KEANOX protein, or antisense nucleic acids complementary to a nucleic acid comprising a KEANOX nucleic acid sequence are additionally provided.

In one embodiment, an antisense nucleic acid molecule is antisense to a “coding region” of the coding strand of a nucleotide sequence encoding KEANOX. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding KEANOX. The term “noncoding region” refers to 5′ and 3′ sequences which flank the coding region that are not translated into amino acids (i.e., also referred to as 5′ and 3′ untranslated regions).

Given the coding strand sequences encoding KEANOX disclosed herein, antisense nucleic acids of the invention can be designed according to the rules of Watson and Crick or Hoogsteen base pairing. The antisense nucleic acid molecule can be complementary to the entire coding region of KEANOX mRNA, but more preferably is an oligonucleotide that is antisense to only a portion of the coding or noncoding region of KEANOX mRNA. For example, the antisense oligonucleotide can be complementary to the region surrounding the translation start site of KEANOX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a KEANOX protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An -anomeric nucleic acid molecule forms specific double-stranded hybrids with complementary RNA in which, contrary to the usual β-units, the strands run parallel to each other (Gaultier et al. (1987) Nucleic Acids Res 15: 6625-6641). The antisense nucleic acid molecule can also comprise a 2′-o-methylribonucleotide (Inoue et al. (1987) Nucleic Acids Res 15: 6131-6148) or a chimeric RNA-DNA analogue (Inoue et al. (1987) FEBS Lett 215: 327-330).

Ribozymes and PNA Moieties

In still another embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach (1988) Nature 334: 585-591)) can be used to catalytically cleave KEANOX mRNA transcripts to thereby inhibit translation of KEANOX mRNA. A ribozyme having specificity for a KEANOX-encoding nucleic acid can be designed based upon the nucleotide sequence of a KEANOX DNA disclosed herein. For example, a derivative of a Tetrahymena L-19 IVS RNA can be constructed in which the nucleotide sequence of the active site is complementary to the nucleotide sequence to be cleaved in a KEANOX-encoding mRNA. See, e.g., Cech et al. U.S. Pat. No. 4,987,071; and Cech et al. U.S. Pat. No. 5,116,742. Alternatively, KEANOX mRNA can be used to select a catalytic RNA having a specific ribonuclease activity from a pool of RNA molecules. See, e.g., Bartel et al., (1993) Science 261: 1411-1418.

Alternatively, KEANOX gene expression can be inhibited by targeting nucleotide sequences complementary to the regulatory region of a KEANOX nucleic acid (e.g., the KEANOX promoter and/or enhancers) to form triple helical structures that prevent transcription of the KEANOX gene in target cells. See generally, Helene. (1991) Anticancer Drug Des. 6: 569-84; Helene. et al. (1992) Ann. N.Y. Acad. Sci. 660: 27-36; and Maher (1992) Bioassays 14: 807-15.

In various embodiments, the nucleic acids of KEANOX can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorg Med Chem 4: 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) above; Perry-O'Keefe et al. (1996) PNAS 93: 14670-675.

PNAs of KEANOX can be used in therapeutic and diagnostic applications. For example, PNAs can be used as antisense or antigene agents for sequence-specific modulation of gene expression by, e.g., inducing transcription or translation arrest or inhibiting replication. PNAs of KEANOX can also be used, e.g., in the analysis of single base pair mutations in a gene by, e.g., PNA directed PCR clamping; as artificial restriction enzymes when used in combination with other enzymes, e.g., S1 nucleases (Hyrup B. (1996) above); or as probes or primers for DNA sequence and hybridization (Hyrup et al. (1996), above; Perry-O'Keefe (1996), above).

In another embodiment, PNAs of KEANOX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of KEANOX can be generated that may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996) above). The synthesis of PNA-DNA chimeras can be performed as described in Hyrup (1996) above and Finn et al. (1996) Nucl Acids Res 24: 3357-63. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA (Mag et al. (1989) Nucl Acid Res 17: 5973-88). PNA monomers are then coupled in a stepwise manner to produce a chimeric molecule with a 5′ PNA segment and a 3′ DNA segment (Finn et al. (1996) above). Alternatively, chimeric molecules can be synthesized with a 5′ DNA segment and a 3′ PNA segment. See, Petersen et al. (1975) Bioorg Med Chem Lett 5: 1119-11124.

In other embodiments, the oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86: 6553-6556; Lemaitre et al., 1987, Proc. Natl. Acad. Sci. 84: 648-652; PCT Publication No. W088/09810) or the blood-brain barrier (see, e.g., PCT Publication No. W089/10134). In addition, oligonucleotides can be modified with hybridization triggered cleavage agents (See, e.g., Krol et al., 1988, BioTechniques 6: 958-976) or intercalating agents. (See, e.g., Zon, 1988, Pharm. Res. 5: 539-549). To this end, the oligonucleotide may be conjugated to another molecule, e.g., a peptide, a hybridization triggered cross-linking agent, a transport agent, a hybridization-triggered cleavage agent, etc.

KEANOX Polypeptides

One aspect of the invention pertains to isolated KEANOX proteins, and biologically active portions thereof, or derivatives, fragments, analogs or homologs thereof. Also provided are polypeptide fragments suitable for use as immunogens to raise anti-KEANOX antibodies. In one embodiment, native KEANOX proteins can be isolated from cells or tissue sources by an appropriate purification scheme using standard protein purification techniques. In another embodiment, KEANOX proteins are produced by recombinant DNA techniques. Alternative to recombinant expression, a KEANOX protein or polypeptide can be synthesized chemically using standard peptide synthesis techniques.

An “isolated” or “purified” protein or biologically active portion thereof is substantially free of cellular material or other contaminating proteins from the cell or tissue source from which the KEANOX protein is derived, or substantially free from chemical precursors or other chemicals when chemically synthesized. The language “substantially free of cellular material” includes preparations of KEANOX protein in which the protein is separated from cellular components of the cells from which it is isolated or recombinantly produced. In one embodiment, the language “substantially free of cellular material” includes preparations of KEANOX protein having less than about 30% (by dry weight) of non-KEANOX protein (also referred to herein as a “contaminating protein”), more preferably less than about 20% of non-KEANOX protein, still more preferably less than about 10% of non-KEANOX protein, and most preferably less than about 5% non-KEANOX protein. When the KEANOX protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of KEANOX protein in which the protein is separated from chemical precursors or other chemicals that are involved in the synthesis of the protein. In one embodiment, the language “substantially free of chemical precursors or other chemicals” includes preparations of KEANOX protein having less than about 30% (by dry weight) of chemical precursors or non-KEANOX chemicals, more preferably less than about 20% chemical precursors or non-KEANOX chemicals, still more preferably less than about 10% chemical precursors or non-KEANOX chemicals, and most preferably less than about 5% chemical precursors or non-KEANOX chemicals.

Biologically active portions of a KEANOX protein include peptides comprising amino acid sequences sufficiently homologous to or derived from the amino acid sequence of the KEANOX protein, e.g., the amino acid sequence encoded by a nucleic acid comprising KEANOX 1-20 that include fewer amino acids than the full length KEANOX proteins, and exhibit at least one activity of a KEANOX protein. Typically, biologically active portions comprise a domain or motif with at least one activity of the KEANOX protein. A biologically active portion of a KEANOX protein can be a polypeptide which is, for example, 10, 25, 50, 100 or more amino acids in length.

A biologically active portion of a KEANOX protein of the present invention may contain at least one of the above-identified domains conserved between the KEANOX proteins. An alternative biologically active portion of a KEANOX protein may contain at least two of the above-identified domains. Another biologically active portion of a KEANOX protein may contain at least three of the above-identified domains. Yet another biologically active portion of a KEANOX protein of the present invention may contain at least four of the above-identified domains.

Moreover, other biologically active portions, in which other regions of the protein are deleted, can be prepared by recombinant techniques and evaluated for one or more of the functional activities of a native KEANOX protein.

In some embodiments, the KEANOX protein is substantially homologous to one of these KEANOX proteins and retains its the functional activity, yet differs in amino acid sequence due to natural allelic variation or mutagenesis, as described in detail below.

In specific embodiments, the invention includes an isolated polypeptide comprising an amino acid sequence that is 80% or more identical to the sequence of a polypeptide whose expression is modulated in a cell to which integrin modulating agent is administered.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See Needleman and Wunsch 1970 J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of a DNA sequence comprising KEANOX: 1-25.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides KEANOX chimeric or fusion proteins. As used herein, an KEANOX “chimeric protein” or “fusion protein” comprises an KEANOX polypeptide operatively linked to a non-KEANOX polypeptide. A “KEANOX polypeptide” refers to a polypeptide having an amino acid sequence corresponding to KEANOX, whereas a “non-KEANOX polypeptide” refers to a polypeptide having an amino acid sequence corresponding to a protein that is not substantially homologous to the KEANOX protein, e g., a protein that is different from the KEANOX protein and that is derived from the same or a different organism. Within a KEANOX fusion protein the KEANOX polypeptide can correspond to all or a portion of a KEANOX protein. In one embodiment, a KEANOX fusion protein comprises at least one biologically active portion of a KEANOX protein. In another embodiment, a KEANOX fusion protein comprises at least two biologically active portions of a KEANOX protein. In yet another embodiment, a KEANOX fusion protein comprises at least three biologically active portions of a KEANOX protein. Within the fusion protein, the term “operatively linked” is intended to indicate that the KEANOX polypeptide and the non-KEANOX polypeptide are fused in-frame to each other. The non-KEANOX polypeptide can be fused to the N-terminus or C-terminus of the KEANOX polypeptide.

For example, in one embodiment a KEANOX fusion protein comprises a KEANOX domain operably linked to the extracellular domain of a second protein. Such fusion proteins can be further utilized in screening assays for compounds which modulate KEANOX activity (such assays are described in detail below).

In yet another embodiment, the fusion protein is a GST-KEANOX fusion protein in which the KEANOX sequences are fused to the C-terminus of the GST (i.e., glutathione S-transferase) sequences. Such fusion proteins can facilitate the purification of recombinant KEANOX.

In another embodiment, the fusion protein is a KEANOX protein containing a heterologous signal sequence at its N-terminus. For example, a native KEANOX signal sequence can be removed and replaced with a signal sequence from another protein. In certain host cells (e.g., mammalian host cells), expression and/or secretion of KEANOX can be increased through use of a heterologous signal sequence.

In yet another embodiment, the fusion protein is a KEANOX-immunoglobulin fusion protein in which the KEANOX sequences comprising one or more domains are fused to sequences derived from a member of the immunoglobulin protein family. The KEANOX-immunoglobulin fusion proteins of the invention can be incorporated into pharmaceutical compositions and administered to a subject to inhibit an interaction between a KEANOX ligand and a KEANOX protein on the surface of a cell, to thereby suppress KEANOX-mediated signal transduction in vivo. The KEANOX-immunoglobulin fusion proteins can be used to affect the bioavailability of a KEANOX cognate ligand. Inhibition of the KEANOX ligand/KEANOX interaction may be useful therapeutically for both the treatments of proliferative and differentiative disorders, as well as modulating (e.g. promoting or inhibiting) cell survival. Moreover, the KEANOX-immunoglobulin fusion proteins of the invention can be used as immunogens to produce anti-KEANOX antibodies in a subject, to purify KEANOX ligands, and in screening assays to identify molecules that inhibit the interaction of KEANOX with a KEANOX ligand.

A KEANOX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An KEANOX-encoding nucleic acid can be cloned into such an expression vector such that the fusion moiety is linked in-frame to the KEANOX protein.

KEANOX Agonists and Antagonists

The present invention also pertains to variants of the KEANOX proteins that function as either KEANOX agonists (mimetics) or as KEANOX antagonists. Variants of the KEANOX protein can be generated by mutagenesis, e.g., discrete point mutation or truncation of the KEANOX protein. An agonist of the KEANOX protein can retain substantially the same, or a subset of, the biological activities of the naturally occurring form of the KEANOX protein. An antagonist of the KEANOX protein can inhibit one or more of the activities of the naturally occurring form of the KEANOX protein by, for example, competitively binding to a downstream or upstream member of a cellular signaling cascade which includes the KEANOX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the KEANOX proteins.

Variants of the KEANOX protein that function as either KEANOX agonists (mimetics) or as KEANOX antagonists can be identified by screening combinatorial libraries of mutants, e.g., truncation mutants, of the KEANOX protein for KEANOX protein agonist or antagonist activity. In one embodiment, a variegated library of KEANOX variants is generated by combinatorial mutagenesis at the nucleic acid level and is encoded by a variegated gene library.

A variegated library of KEANOX variants can be produced by, for example, enzymatically ligating a mixture of synthetic oligonucleotides into gene sequences such that a degenerate set of potential KEANOX sequences is expressible as individual polypeptides, or alternatively, as a set of larger fusion proteins (e.g., for phage display) containing the set of KEANOX sequences therein. There are a variety of methods which can be used to produce libraries of potential KEANOX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential KEANOX sequences. Methods for synthesizing degenerate oligonucleotides are known in the art (see, e.g., Narang (1983) Tetrahedron 39: 3; Itakura et al. (1984) Annu Rev Biochem 53: 323; Itakura et al. (1984) Science 198: 1056; Ike et al. (1983) Nucl Acid Res 11: 477.

Polypeptide Libraries

In addition, libraries of fragments of the KEANOX protein coding sequence can be used to generate a variegated population of KEANOX fragments for screening and subsequent selection of variants of a KEANOX protein. In one embodiment, a library of coding sequence fragments can be generated by treating a double stranded PCR fragment of a KEANOX coding sequence with a nuclease under conditions wherein nicking occurs only about once per molecule, denaturing the double stranded DNA, renaturing the DNA to form double stranded DNA that can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the KEANOX protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of KEANOX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify KEANOX variants (Arkin and Yourvan (1992) PNAS 89: 7811-7815; Delgrave et al. (1993) Protein Engineering 6: 327-331).

Anti-KEANOX Antibodies

An isolated KEANOX protein, or a portion or fragment thereof, can be used as an immunogen to generate antibodies that bind KEANOX using standard techniques for polyclonal and monoclonal antibody preparation. The full-length KEANOX protein can be used or, alternatively, the invention provides antigenic peptide fragments of KEANOX for use as immunogens. The antigenic peptide of KEANOX comprises at least 8 amino acid residues of the amino acid sequence encoded by a nucleic acid comprising the nucleic acid sequence shown in KEANOX: 1-25 and encompasses an epitope of KEANOX such that an antibody raised against the peptide forms a specific immune complex with KEANOX. Preferably, the antigenic peptide comprises at least 10 amino acid residues, more preferably at least 15 amino acid residues, even more preferably at least 20 amino acid residues, and most preferably at least 30 amino acid residues. Preferred epitopes encompassed by the antigenic peptide are regions of KEANOX that are located on the surface of the protein, e.g., hydrophilic regions. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety.

KEANOX polypeptides or derivatives, fragments, analogs or homologs thereof, may be utilized as immunogens in the generation of antibodies that immunospecifically-bind these protein components. The term “antibody” as used herein refers to immunoglobulin molecules and immunologically active portions of immunoglobulin molecules, i.e., molecules that contain an antigen binding site that specifically binds (immunoreacts with) an antigen. Such antibodies include, but are not limited to, polyclonal, monoclonal, chimeric, single chain, F_{ab} and F_{(ab′)2} fragments, and an F_{ab} expression library. Various procedures known within the art may be used for the production of polyclonal or monoclonal antibodies to a KEANOX protein sequence, or derivatives, fragments, analogs or homologs thereof. Some of these proteins are discussed below.

For the production of polyclonal antibodies, various suitable host animals (e.g., rabbit, goat, mouse or other mammal) may be immunized by injection with the native protein, or a synthetic variant thereof, or a derivative of the foregoing. An appropriate immunogenic preparation can contain, for example, recombinantly expressed KEANOX protein or a chemically synthesized KEANOX polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against KEANOX can be isolated from the mammal (e.g., from the blood) and further purified by well known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of KEANOX. A monoclonal antibody composition thus typically displays a single binding affinity for a particular KEANOX protein with which it immunoreacts. For preparation of monoclonal antibodies directed towards a particular KEANOX protein, or derivatives, fragments, analogs or homologs thereof, any technique that provides for the production of antibody molecules by continuous cell line culture may be utilized. Such techniques include, but are not limited to, the hybridoma technique (see Kohler & Milstein, 1975 Nature 256: 495-497); the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al, 1985 In: MONOCLONAL ANTIBODIES AND CANCER THERAPY, Alan R. Liss, Inc., pp. 77-96).

According to the invention, techniques can be adapted for the production of single-chain antibodies specific to a KEANOX protein (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a KEANOX protein or derivatives, fragments, analogs or homologs thereof. Non-human antibodies can be “humanized” by techniques well known in the art. See e.g., U.S. Pat. No. 5,225,539. Antibody fragments that contain the idiotypes to a KEANOX protein may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Additionally, recombinant anti-KEANOX antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT International Application No. PCT/US86/02269; European Patent Application No. 184,187; European Patent Application No. 171,496; European Patent Application No. 173,494; PCT International Publication No. WO 86/01533; U.S. Pat. No. 4,816,567; European Patent Application No. 125,023; Better et al. (1988) Science 240: 1041-1043; Liu et al. (1987) PNAS 84: 3439-3443; Liu et al. (1987) J Immunol. 139: 3521-3526; Sun et al. (1987) PNAS 84: 214-218; Nishimura et al. (1987) Cancer Res 47: 999-1005; Wood et al. (1985) Nature 314: 446-449; Shaw et al. (1988) J Natl Cancer Inst. 80: 1553-1559); Morrison (1985) Science 229: 1202-1207; Oi et al. (1986) BioTechniques 4: 214; U.S. Pat. No. 5,225,539; Jones et al. (1986) Nature 321: 552-525; Verhoeyan et al. (1988) Science 239: 1534; and Beidler et al. (1988) J Immunol 141: 4053-4060.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of a KEANOX protein is facilitated by generation of hybridomas that bind to the fragment of a KEANOX protein possessing such a domain. Antibodies that are specific for one or more domains within a KEANOX protein, e.g., domains spanning the above-identified conserved regions of KEANOX family proteins, or derivatives, fragments, analogs or homologs thereof, are also provided herein.

Anti-KEANOX antibodies may be used in methods known within the art relating to the localization and/or quantitation of a KEANOX protein (e.g., for use in measuring levels of the KEANOX protein within appropriate physiological samples, for use in diagnostic methods, for use in imaging the protein, and the like). In a given embodiment, antibodies for KEANOX proteins, or derivatives, fragments, analogs or homologs thereof, that contain the antibody derived binding domain, are utilized as pharmacologically-active compounds [hereinafter “Therapeutics”].

An anti-KEANOX antibody (e.g., monoclonal antibody) can be used to isolate KEANOX by standard techniques, such as affinity chromatography or immunoprecipitation. An anti-KEANOX antibody can facilitate the purification of natural KEANOX from cells and of recombinantly produced KEANOX expressed in host cells. Moreover, an anti-KEANOX antibody can be used to detect KEANOX protein (e.g., in a cellular lysate or cell supernatant) in order to evaluate the abundance and pattern of expression of the KEANOX protein. Anti-KEANOX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

KEANOX Recombinant Expression Vectors and Host Cells

Another aspect of the invention pertains to vectors, preferably expression vectors, containing a nucleic acid encoding KEANOX protein, or derivatives, fragments, analogs or homologs thereof. As used herein, the term “vector” refers to a nucleic acid molecule capable of transporting another nucleic acid to which it has been linked. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal manmnalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively linked to the nucleic acid sequence to be expressed. Within a recombinant expression vector, “operably linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., KEANOX proteins, mutant forms of KEANOX, fusion proteins, etc.).

The recombinant expression vectors of the invention can be designed for expression of KEANOX in prokaryotic or eukaryotic cells. For example, KEANOX can be expressed in bacterial cells such as E. coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (1) to increase expression of recombinant protein; (2) to increase the solubility of the recombinant protein; and (3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69: 301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (Wada et al., (1992) Nucleic Acids Res. 20: 2111-2118). Such alteration of nucleic acid sequences of the invention can be carried out by standard DNA synthesis techniques.

In another embodiment, the KEANOX expression vector is a yeast expression vector. Examples of vectors for expression in yeast S. cerevisiae include pYepSec1 (Baldari, et al., (1987) EMBO J 6: 229-234), pMFa (Kurjan and Herskowitz, (1982) Cell 30: 933-943), pJRY88 (Schultz et al., (1987) Gene 54: 113-123), pYES2 (Invitrogen Corporation, San Diego, Calif.), and picZ (InVitrogen Corp, San Diego, Calif.).

Alternatively, KEANOX can be expressed in insect cells using baculovirus expression vectors. Baculovirus vectors available for expression of proteins in cultured insect cells (e.g., SF9 cells) include the pAc series (Smith et al. (1983) Mol Cell Biol 3: 2156-2165) and the pVL series (Lucklow and Summers (1989) Virology 170: 31-39).

In yet another embodiment, a nucleic acid of the invention is expressed in mammalian cells using a mammalian expression vector. Examples of mammalian expression vectors include pCDM8 (Seed (1987) Nature 329: 840) and pMT2PC (Kaufman et al. (1987) EMBO J 6: 187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells. See, e.g., Chapters 16 and 17 of Sambrook et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev 1: 268-277), lymphoid-specific promoters (Calame and Eaton (1988) Adv Immunol 43: 235-275), in particular promoters of T cell receptors (Winoto and Baltimore (1989) EMBO J 8: 729-733) and immunoglobulins (Banerji et al. (1983) Cell 33: 729-740; Queen and Baltimore (1983) Cell 33: 741-748), neuron-specific promoters (e.g., the neurofilament promoter; Byrne and Ruddle (1989) PNAS 86: 5473-5477), pancreas-specific promoters (Edlund et al. (1985) Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss (1990) Science 249: 374-379) and the fetoprotein promoter (Campes and Tilghman (1989) Genes Dev 3: 537-546).

The invention further provides a recombinant expression vector comprising a DNA molecule of the invention cloned into the expression vector in an antisense orientation. That is, the DNA molecule is operatively linked to a regulatory sequence in a manner that allows for expression (by transcription of the DNA molecule) of an RNA molecule that is antisense to KEANOX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, KEANOX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding KEANOX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) a KEANOX protein. Accordingly, the invention further provides methods for producing KEANOX protein using the host cells of the invention. In one embodiment, the method comprises culturing the host cell of invention (into which a recombinant expression vector encoding KEANOX has been introduced) in a suitable medium such that KEANOX protein is produced. In another embodiment, the method further comprises isolating KEANOX from the medium or the host cell.

Pharmaceutical Compositions

The KEANOX nucleic acid molecules, KEANOX proteins, and anti-KEANOX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic. Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a KEANOX protein or anti-KEANOX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery. In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) PNAS 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system. The pharmaceutical compositions can be included in a container, pack, or dispenser together with instructions for administration.

Kits and Nucleic Acid Collections for Identifying KEANOX Nucleic Acids

In another aspect, the invention provides a kit useful for examining integrin modulating agents. The kit can include nucleic acids that detect two or more KEANOX sequences. In preferred embodiments, the kit includes reagents which detect 3, 4, 5, 6, 8, 10, 12, 15, 20, 25, 50, 100, 150, 200 or all of the KEANOX nucleic acid sequences.

The invention also includes an isolated plurality of sequences which can identify one or more KEANOX responsive nucleic acid sequences.

The kit or plurality may include, e.g., sequence homologous to KEANOX nucleic acid sequences, or sequences which can specifically identify one or more KEANOX nucleic acid sequences.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of identifying a integrin modulating agent, the method comprising;
(a) providing a test cell population comprising a cell capable of expressing one or more nucleic acid sequences selected from the group consisting of KEANOX1-259 and 260;
(b) contacting the test cell population with a test agent;
(c) measuring expression of one or more of the nucleic acid sequences in the test cell population;
(d) comparing the expression of the nucleic acid sequences in the test cell population to the expression of the nucleic acid sequences in a reference cell population comprising at least one cell whose integrin modulating agent expression status is known; and
(e) identifying a difference in expression levels of the KEANOX sequence, if present, in the test cell population and reference cell population, thereby identifying an integrin modulating agent

2. The method of claim 1, wherein the method comprises comparing the expression of 200 or more of the nucleic acid sequences.

3. The method of claim 1, wherein the method comprises comparing the expression of 100 or more of the nucleic acid sequences.

4. The method of claim 1, wherein the method comprises comparing the expression of 25 or more of the nucleic acid sequences.

5. The method of claim 1, wherein the expression of the nucleic acid sequences in the test cell population is decreased as compared to the reference cell population.

6. The method of claim 1, wherein the expression of the nucleic acid sequences in the test cell population is increased as compared to the reference cell population.

7. The method of claim 1, wherein the test cell population is provided in vitro.

8. The method of claim 1, wherein the test cell population is provided ex vivo from a mammalian subject.

9. The method of claim 1, wherein the test cell population is provided in vivo in a mammalian subject.

10. The method of claim 1, wherein the test cell population is derived from a human or rodent subject.

11. The method of claim 1, wherein the test cell population includes a blood cell.

12. The method of claim 1, wherein the test cell population includes a monocyte.

13. An isolated nucleic acid comprising a nucleic acid sequence selected from the group consisting of a KEANOX 1-25 nucleic acid, or its complement.

14. A vector comprising the nucleic acid of claim 13.

15. A cell comprising the vector of claim 14.

16. A pharmaceutical composition comprising the nucleic acid of claim 13.

17. A polypeptide encoded by the nucleic acid of claim 13.

18. An antibody which specifically binds to the polypeptide of claim 17.

19. A kit which detects two or more of the nucleic acid sequences selected from the group consisting of KEANOX: 1-260.

20. An array which detects one or more of the nucleic acid selected from the group consisting of KEANOX: 1-260.

21. A plurality of nucleic acid comprising one or more of the nucleic acid selected from the group consisting of KEANOX: 1-260.
